(19) United States
(12) Patent Application Publication
(10) Pub. No.: US 2025/0243139 A1
(43) Pub. Date: Jul. 31, 2025

(54) THERMAL DECOMPOSITION OF SODIUM FORMATE AND SODIUM OXALATE USING SUPER-HEATED STEAM FROM NUCLEAR REACTOR SYSTEM FOR DIRECT IN-SITU METHANOL PRODUCTION

(71) Applicant: NuScale Power, LLC, Corvallis, OR (US)

(72) Inventors: Francis Y. Tsang, Bellingham, WA (US); José N. Reyes, JR., Corvallis, OR (US); Luis DePavia, Corvallis, OR (US)

(73) Assignee: NuScale Power, LLC, Corvallis, OR (US)

(21) Appl. No.: 19/036,796

(22) Filed: Jan. 24, 2025

Related U.S. Application Data
(60) Provisional application No. 63/625,284, filed on Jan. 26, 2024.

(51) Int. Cl. C07C 29/151; C01D 1/04; C07C 51/41; G21C 1/32; G21D 9/00; H02J 3/46

(52) U.S. Cl. CPC C07C 29/1518; C01D 1/04; C07C 51/418; G21D 9/00; H02J 3/46; G21C 1/32

(57) ABSTRACT

An integrated energy system including a power plant is discussed herein. In some examples, the integrated energy system may include at least one nuclear reactor and electrical power generation system configured to generate steam and electricity, a water treatment plant configured to produce Sodium Hydroxide (NaOH) from salt water, a Sodium Formate (HCOONa) production plant configured to receive the Sodium Hydroxide (NaOH) to produce Sodium Formate (HCOONa), a Thermal Decomposition reactor configured to receive the Sodium Formate (HCOONa) and configured to receive at least a first portion of the steam or at least a second portion of the electricity from the power plant to indirectly heat the Thermal Decomposition reactor to produce Hydrogen (H₂), Carbon Dioxide (CO₂), and Carbon Monoxide (CO) from the Sodium Formate (HCOONa), and a Methanol (CH₃OH) reaction chamber configured to receive the Hydrogen (H₂), the Carbon Dioxide (CO₂), and the Carbon Monoxide (CO) to produce Methanol (CH₃OH).

### CROSS-REFERENCE TO RELATED APPLICATIONS

This application claims the benefit of U.S. Provisional Patent Application No. 63/625,284 filed Jan. 26, 2024, and titled “THERMAL DECOMPOSITION OF SODIUM FORMATE AND SODIUM OXALATE USING SUPER-HEATED STEAM FROM NUCLEAR REACTOR SYSTEM FOR DIRECT IN-SITU METHANOL PRODUCTION,” which is incorporated herein by reference in its entirety.

### TECHNICAL FIELD

The present technology is directed to nuclear reactor integrated energy systems (IESs) for energy production and green industrial applications, such as to integrated energy systems including one or more nuclear reactors (e.g., small modular nuclear reactors (SMRs)) coupled to a desalination plant for simultaneously producing hydrogen and chlorine gases, capturing carbon dioxide gas, and the regeneration of carbon dioxide and carbon monoxide gases for methanol production.

### BACKGROUND

Modern society depends on a steady supply of electrical energy from a power grid whenever electrical energy is needed. Accordingly, a power grid requires a dependable source of electrical energy from energy producers in order to provide consistent electrical energy to consumers, whenever electrical energy is needed. Depending on the geographic location, population, and other factors, demand for electrical energy may be relatively high at certain times of the day and relatively lower at other times. For example, highly populated areas with hot climates may experience a large increase in energy demand in the evenings caused by a large number of consumers getting home from work and turning on air conditioning units. Similarly, the same highly populated areas with hot climates may see large decreases in energy demand in the evenings caused by the large number of consumers turning off air conditioners and going to bed. The times of high energy demand, or “peak times,” and times of low energy demand, or “off-peak times,” may be anticipated and planned for. As a result of the fluctuations between peak times and off-peak times, energy providers (e.g., nuclear, solar, natural gas, fossil fuel, etc.) may experience similar fluctuations in the demand for energy production (i.e., high energy production demand during peak times is greater than during off-peak times).

An energy imbalance market (“EIM”) is a means of supplying energy when and where it is needed to balance fluctuations in energy demand (i.e., peak times vs. off-peak times) and subsequent fluctuations in energy production demand (i.e., energy production demand during peak times vs. energy production demand during off-peak times). To increase productivity and efficiency for energy producers experiencing fluctuating energy production demand, new processes, systems, and methods are needed.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures may indicate similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

FIG. 1 schematically illustrates a representation of an integrated energy system (IES) 100 that includes a small modular nuclear reactor (SMR) system integrated with a Methanol (CH₃OH) production system.

FIGS. 2A-D illustrate representative schematic diagrams of Thermal Decomposition reactor systema for the production of Methanol (CH₃OH) during steady state operations.

FIG. 3 schematically illustrates a representation of an integrated energy system (IES) 300 that includes a small modular nuclear reactor (SMR) system integrated with a Hydrogen (H₂) production system and a Hydrogen Fuel Cell.

FIG. 4 illustrates a steady-state Hydrogen (H₂) production process utilizing Sodium Formate (HCOONa) and a Hydrogen Fuel Cell.

FIG. 5 illustrates in-situ and on-demand Hydrogen (H₂) production system to support emergency and limited energy imbalance market (EIM) using Sodium Formate (HCOONa).

FIG. 6 schematically illustrates a representative schematic diagram of a Hydrogen (H₂) extraction reactor during steady state operations.

FIG. 7A schematically illustrates a representative schematic diagram of a Hydrogen (H₂) extraction reactor during steady state operations for the thermal decomposition of Sodium Formate (HCOONa) (i.e., dry process).

FIG. 7B schematically illustrates a representative schematic diagram of a Hydrogen (H₂) extraction reactor during steady state operations for the hydrothermal decomposition of Sodium Formate (HCOONa) (i.e., wet process).

FIG. 8 illustrates a Hydrogen (H₂) production process using multiple Sodium Formate (HCOONa) production systems simultaneously.

FIG. 9 illustrates an integrated energy system (IES) configured to capture atmospheric Carbon Dioxide (CO₂) to produce Carbon Dioxide (CO₂) and Carbon Monoxide (CO) for use in Methanol (CH₃OH) production.

FIG. 10 illustrates a flow diagram of an example process associated with utilizing a Hydrogen (H₂) extraction reactor and a solid oxide electrolysis cell (SOEC) for the simultaneous production of Sodium Oxalate ((COO)₂Na₂) and Formaldehyde (CH₂O).

FIG. 11 illustrates a flowchart describing an example process for utilizing a power plant system to produce Hydrogen (H₂) and Methanol (CH₃OH) which can be used in an EIM.

FIG. 12 is a partially schematic, partially cross-sectional view of a nuclear reactor system configured in accordance with embodiments of the present technology.

FIG. 13 is a partial schematic, partial cross-sectional view of a nuclear reactor system configured in accordance with additional embodiments of the present technology.

FIG. 14 is a schematic view of a nuclear power plant system including multiple nuclear reactors in accordance with embodiments of the present technology.

### DETAILED DESCRIPTION

This disclosure is directed to an integrated small modular nuclear reactor (SMR) system that can be emplaced as a baseload energy generator to produce electricity for the power grid during periods where energy production demand is high (“peak times”), such as to generate electricity to support an EIM. In some instances, the EIM time slot may typically be defined between 6:00 p.m. to 10:00 p.m. (about a 4-hour period). During the period when the demand for electricity is low or below the typical baseload supply (“off-peak times”), then some of the reactor systems (e.g., SMRs) can be utilized to provide electricity and steam to produce Hydrogen (H₂), Carbon Dioxide (CO₂), and Carbon Monoxide (CO), such as through Thermal Decomposition of Sodium Formate (HCOONa), for the production of Methanol (CH₃OH).

In embodiments, the present disclosure is directed to techniques that may be performed in relation to Integrated Energy Systems (IESs), such as for use in green industrial processes that produce few or no carbon emissions, to capture carbon from an emission source, for resource production, and associated devices and methods. IESs of the present technology may include a power plant (e.g., a primary power plant) that is integrated with one or more industrial processes and resource production plants to provide power with few or no carbon emissions, to produce resources, such as clean water, Sodium Hydroxide (NaOH), Hydrogen (H₂), Chlorine (Cl₂), Carbon Monoxide (CO), Carbon Dioxide (CO₂), Sodium Formate (NaHCOO), Sodium Oxalate ((COO)₂Na₂), Sodium Carbonate (Na₂CO₃), Sodium Oxide (Na₂O), and Methanol (CH₃OH).

Industrial processes in accordance with embodiments of the present technology may include water purification, chemical manufacturing and production, natural-gas or coal-fired power generation plants, petroleum and oil refining, bulk plastic waste recycling and gasification, cement production, ore processing plants, steel and primary metal manufacturing, transportation, food processing, pharmaceutical production, pulp and paper, materials manufacturing, and/or other industrial plants. Such an IES may be capable of providing electricity and steam, or a combination of both, from the power plant to the industrial processes for carbon capture and resource production, such as the production of chemical products. The IES of the present disclosure can also assist industries to meet EPA and other national and global regulations for cutting Carbon Dioxide (CO₂) emissions. In embodiments, the IES may be modular and therefore may be retrofit to existing industrial processes for power supply, steam supply, and/or resource production.

Because of the drive toward cleaner and more efficient forms of power production, nuclear power will be increasingly important in the coming years. In operation, nuclear power plants use the nuclear fission process to generate heat, which is then used to produce steam to turn turbines and generate electricity. This process can result in the production of electrical power that reduces the need for coal and natural gas to produce electricity. Nuclear power plants provide reliable baseload power without emitting greenhouse gases such as Carbon Dioxide (CO₂) during operation, making them attractive for countries that are seeking to reduce carbon emissions and enhance energy security. Due to the advantages of nuclear energy for providing electricity, the present disclosure presents novel methods of using nuclear power in integrated energy systems for carbon capture and “green” resource production, such as the production of “green” chemical products.

In some embodiments, an IES includes a power plant system having multiple small modular nuclear reactors (SMRs) specifically configured to operate in unison to support one or more of the industrial processes. SMRs are nuclear reactors that are smaller in terms of size (e.g., dimensions) and power compared to large, conventional nuclear reactors. Moreover, they are modular in that some or all of their systems and components can be factory-assembled and transported as a unit to a location for installation. In some aspects of the present technology, the multiple SMRs of the integrated energy system can flexibly and dynamically provide electricity, steam, or a combination of both electricity and steam to the industrial processes due to the modularity and flexibility of the SMRs. That is, a configuration of the SMRs can be switched during operation to provide varying levels of steam and electricity output depending on the operational states and/or demands of the industrial processes.

In embodiments, a power plant of the present disclosure can be a permanent or temporary installation built at or near (e.g., roughly 1 km from) the location of an industrial process facility or can be a mobile or partially mobile system that is moved to and assembled at or near the location of the industrial process facility. More generally, the power plant can be local (e.g., positioned at or near) to the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant is configured to supply a portion of electricity to a power grid.

In some embodiments, the present disclosure includes systems and methods that may address many problems associated with conventional resource production processes, such as reducing carbon emissions and improving economic viability. In embodiments, the IES of the present disclosure may receive supply water, such as saline water from a water source, and produce steam, power, and one or more resources and/or products such as clean water, Sodium Hydroxide (NaOH), Hydrogen (H₂), Chlorine (Cl₂), Carbon Monoxide (CO), Carbon Dioxide (CO₂), Sodium Formate (NaHCOO), Sodium Oxalate ((COO)₂Na₂), Sodium Carbonate (Na₂CO₃), Sodium Oxide (Na₂O), and Methanol (CH₃OH). In embodiments, resources and/or products produced in the IES may be sold directly to industry. In embodiments, resources and/or products within the IES may be further utilized in the production of other resources and/or products that can be used within the IES or sold to industry.

In some embodiments, the IESs may be configured to produce Methanol (CH₃OH). Methanol (CH₃OH), also known as methyl alcohol, is a highly versatile chemical widely used for industrial purposes and prevalent in our everyday lives. It is a base material in the production of acetic acid and formaldehyde, and also increasingly being used in ethylene and propylene production. Methanol (CH₃OH) is one of the most prolific intermediate materials for the production of other chemicals and materials. In the chemical industry Methanol (CH₃OH) mainly serves as a raw material in the production of formaldehyde, olefins, acetic acid, MTBE, DME, as well as biodiesel. So, renewable Methanol (CH₃OH) is a pre-requisite for making a broad range of chemical products green such as polymer fibers for the textile industry, plastics for packaging, glues, adsorbents/diapers, paints, adhesives, solvents, and much more. Besides its use in the chemical, construction and plastics industries, Methanol (CH₃OH) also serves as a fuel or fuel additive. Methanol is an extremely efficient hydrogen carrier because one methanol molecule has more hydrogen atoms than one hydrogen molecule. Methanol is a liquid at ambient conditions. Therefore, methanol can be handled, stored, and transported with ease by leveraging existing industrial infrastructures.

The conventional production method for Methanol (CH₃OH) involves a catalytic process using fossil feedstock such as natural gas, coal, or syngas. Currently, most Methanol (CH₃OH) is produced by hydrogenating Carbon Dioxide (CO₂) with Hydrogen (H₂) with high selectivity on conventional Copper (Cu) and zinc oxide (ZnO) based catalysts (Cu/ZnO) following the reaction in Equation 1:

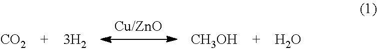

The synthesis of Methanol (CH₃OH) from Carbon Dioxide (CO₂) however, is complicated because of water formation. In the absence of Carbon Monoxide (CO), water is produced as the by-product of Carbon Dioxide (CO₂) hydrogenation. The increased formation of water leads to kinetic inhibition and acerated deactivation of the Cu/ZnO catalysts. To counteract the formation of water and prevent the deactivation of the Cu/ZnO catalysts, Carbon Monoxide (CO) is required in the reaction chamber to remove water via a Water-Gas-Shift (WGS) reaction. Therefore, the introduction of Carbon Monoxide (CO) into the reaction chamber can remove water via the Water-Gas-Shift (WGS) reaction so that the production of Methanol (CH₃OH) can be continued, following the reactions in Equations 2 and 3, where Equation 3 is the WGS reaction:

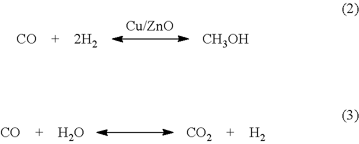

Methanol may also be produced industrially by hydrogenation of Carbon Monoxide (CO) over a catalyst. The most widely used catalyst is a mixture of Copper and Zinc oxides at temperature at about 250° C. and between 5-10 MPa (50-100 atm). The reaction is the same as Equation 2. Note there are two moles of hydrogen per one mole of carbon monoxide in Equation 2. In most industrial production process, Methane (CH₄) gas typically is used to produce syngas—a combination of Hydrogen (H₂) and Carbon Monoxide (CO). The produced syngas contains three moles of hydrogen for every mole of carbon monoxide. Typically, Carbon Dioxide (CO₂) is injected into the synthesis reactor to react with the extra hydrogen to complete the production process, shown in Equation 1.

The predominant method of producing Hydrogen (H₂), Carbon Dioxide (CO₂), and Carbon Monoxide (CO) for Methanol (CH₃OH) production is through the steam-methane reforming of natural gas. The steam-reforming process, however, results in significant Carbon Dioxide (CO₂) emissions into the atmosphere. Currently, there is no existing process that can simultaneously produce all three gases, Hydrogen (H₂), Carbon Dioxide (CO₂), and Carbon Monoxide (CO) for Methanol (CH₃OH) production.

In embodiments of the present disclosure, the IES can produce Hydrogen (H₂), Carbon Monoxide (CO), and Carbon Dioxide (CO₂) for the production of Methanol (CH₃OH). In some embodiments, the IES receives water from a water source, such as an ocean. In embodiments, water from the water source is fed to a water purification plant, such as a Reverse Osmosis (RO) desalination plant, configured to produce clean water.

As climate change progresses, water scarcity will become an increasing threat to nations and individuals throughout the world. Nearly 30% of the human population live in water-stressed countries, and 9% live in critically water-stressed countries. Desalination has been successfully implemented in many regions mitigating the negative effects of water scarcity by providing clean high-quality water for the local populations. Desalination, however, is a resource and energy intensive process with RO, the most commonly used desalination process, requiring between 3.44-22.36 kWh/m³ of freshwater produced. Globally, RO accounts for around 69% of installed desalination capacity or about 69 million m³/day, thus this equates to approximate emissions of 33 billion kg of Carbon Dioxide (CO₂) per year for clean RO water. Therefore, there is a need to develop integrated energy systems that produce few, or no carbon emissions, to reduce the carbon burden of clean water production.

Desalination of seawater also produces large quantities of brine as a by-product. Brine is a high concentration salt and water solution, primarily Sodium Chloride (NaCl) ranging from about 5% to about 26%. Brine is denser than seawater and therefore sinks to the bottom of the ocean and if released directly, can damage ecosystems. The desalination of seawater through RO on average produces about 1.4 liters of brine for every liter of clean water. Or in other words, for RO to produce the 69 million m³/day of fresh water on a global scale, there must be 97 million m³/day of brine that requires proper environmental disposal. Therefore, there is a need to develop integrated energy systems that produce few, or no carbon emissions, that can address the current burden of brine formation in the desalination of sea water.

In embodiments, the present disclosure includes methods and devices that may address many problems associated with conventional clean water production solutions such as the production of clean water with few or no carbon emissions. The treatments of brine as the result of the reverse osmosis process to produce clean water are described in Applicant's U.S. Provisional Patent Application No. 63/507,057, filed Jun. 8, 2023, and entitled “NUCLEAR REACTOR INTEGRATED ENERGY SYSTEMS FOR THE DIRECT CAPTURE OF CARBON DIOXIDE FROM EMISSIONS SOURCES FOR METHANOL PRODUCTION” and U.S. Provisional Patent Application No. 63/602,227, filed Nov. 22, 2023, and entitled “SODIUM FORMATE HYDROGEN EXTRACTION SYSTEM OPERATION AND PRODUCTION OF HYDROGEN AND METHANOL,” each of which is incorporated herein by reference in its entirety.

Treatment of the brine can generate Sodium Hydroxide (NaOH) and Sodium Hydroxide (NaOH) can be converted into Sodium Formate (HCOONa). Sodium Hydroxide (NaOH) solution can also be used to capture carbon dioxide from the ambient air via a Direct Air Capture (DAC) process. Aqueous Sodium Hydroxide (NaOH) solution can chemically react with Carbon Dioxide (CO₂), for example from ambient or a carbon dioxide emission source, to form Sodium Carbonate (Na₂CO₃) and Sodium Bicarbonate (NaHCO₃). The Sodium Carbonate (Na₂CO₃) and Sodium Bicarbonate (NaHCO₃) may then be reacted with a carboxylic acid, such as Formic Acid, to generate Sodium Formate (HCOONa). In embodiments, Sodium Formate (HCOONa) can be produced by neutralizing Formic Acid (HCOOH) with the Sodium Hydroxide (NaOH) solution.

Some embodiments of the techniques described herein include two coupling processes to thermally decompose the Sodium Formate (HCOONa) and its products Sodium Oxalate ((COO)₂Na₂) and Sodium Carbonate (Na₂CO₃), with super-heated steam and compression heating to produce the three gases (Hydrogen (H₂), Carbon Monoxide (CO), and Carbon Dioxide (CO₂)) in a continuous manner that are used for in-situ Methanol (CH₃OH) production. In embodiments, the systems described herein produce negative carbon emissions.

In an exemplary embodiment, Sodium Formate (HCOONa) salt in a first thermal decomposition chamber is thermally decomposed using the super-heated steam generated from a nuclear reactor, such as an SMR according to embodiments described herein, to maintain the temperature in the chamber to be between 360° C. to 400 C. In embodiments, the super-heated steam does not make contact with the Sodium Formate (HCOONa) and this process is a “dry” thermal decomposition process. The decomposition reactions produce Hydrogen (H₂) and Carbon Monoxide (CO) gases and the by-product Sodium Carbonate (Na₂CO₃) in the first thermal decomposition chamber. The reaction equations are:

2HCOONa→(COO)₂Na₂+H₂ (4)

(COO)₂Na₂→Na₂CO₃+CO (5)

The temperature inside the first reaction chamber has to keep constant, for example at 350° C., with non-interrupted supply of super-heated steam from the nuclear reactors. The Sodium Formate (HCOONa) powder after entering the first chamber will be decomposed by the thermal shock wave in the “dry” thermal decomposition process. As a result, the hydrogen gas will be produced instantaneously. The first reaction by-product, Sodium Oxalate ((COO)₂Na₂) Equation 4, also will be thermally decomposed to produce Carbon Monoxide (CO) gas and Sodium Carbonate (Na₂CO₃) as its by-product Equation 5. The generation of Carbon Monoxide (CO) gas is instantaneous because the Sodium Carbonate (Na₂CO₃) is in powder form and the thermal shock will make the reaction instantaneous and complete. The mixture will sink to the bottom of the first reaction chamber while still thermally hot. The mixture is transferred immediately into a second decomposition chamber.

In the second thermal decomposition chamber, the Sodium Carbonate (Na₂CO₃) mixture is fed directly into the second chamber from the bottom of the first decomposition chamber and is instantly heated to >800° C. The second chamber temperature is raised and maintained by the super-heat steam. Again, the super-heated steam is only used to maintain the temperature in the reaction chamber and is not used to interact with the Sodium Carbonate (Na₂CO₃) such that this process is a totally “dry” thermal decomposition process. The reaction produces both Carbon Dioxide (CO₂) and the by-product Sodium Oxide (Na₂O). Since the temperature in the second decomposition may be maintained to >800° C., the Sodium Carbonate (Na₂CO₃) may instantaneously be decomposed to Sodium Oxide (Na₂O) and Carbon Dioxide (CO₂). The two processes can be described by the following equation:

Na₂CO₃→Na₂O+CO₂ (6)

The end product generated from these two “dry” thermal decomposed processes starting with Sodium Formate (HCOONa) is Sodium Oxide (Na₂O). Sodium Oxide can be used to make high quality glass and lens products. The three produced gases, Hydrogen (H₂), Carbon Monoxide (CO), and Carbon Dioxide (CO₂), can be introduced directly into a synthesis chamber for the in-situ Methanol (CH₃OH) production. The prescribed setup is unique and the Methanol (CH₃OH) production is rapid and continuous.

The colocation of the two thermal decomposition chambers of this invention is energy efficient since the Sodium Carbonate (Na₂CO₃) at the output of the first stage is elevated, such as at 350° C., prior to heating up to >800° C. in the second stage. Heating of the thermal decomposition chambers can be done by compression heating of steam. This process is described in Applicant's U.S. Provisional Patent Application No. 63/504,230, FILED May 25, 2023, and entitled “NUCLEAR REACTOR SYSTEMS INCLUDING DIRECT CYCLE WITH COMPRESSION AND PEAKING HEAT” and U.S. Provisional Patent Application No. 63/504,231, filed May 25, 2023, and entitled “NUCLEAR ERACTOR SYSTEMS INCLUDING INDIRECT CYCLE WITH INTERMEDIATE HEAT EXCHANGER, PROCESS HEAT RECOVERY, AND PEAKING HEAT,” each of which is incorporated herein by reference in its entirety.

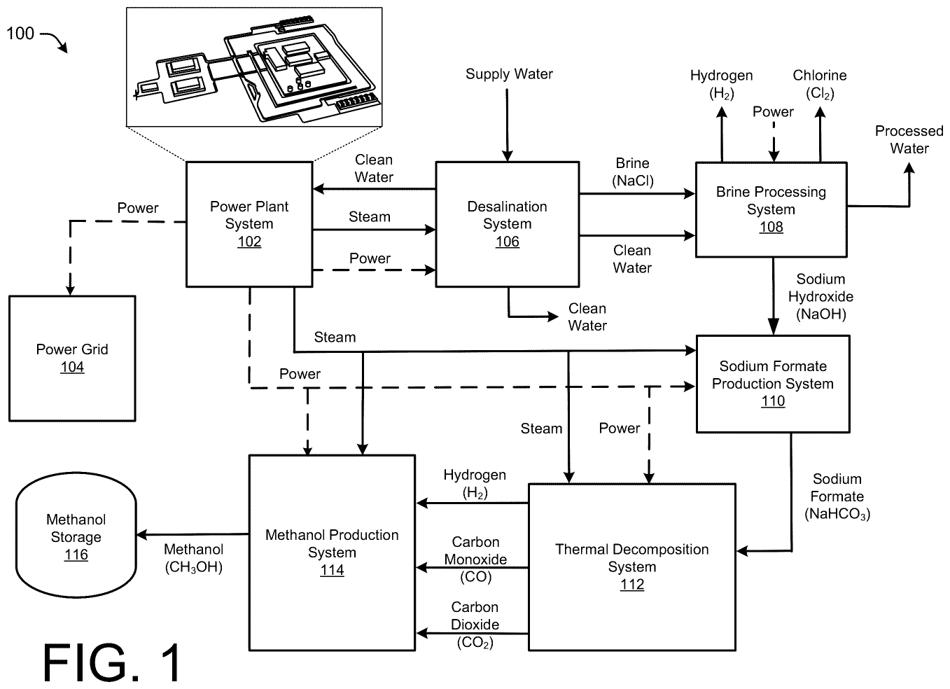

Certain details are set forth in the following description and in FIGS. 1-14 to provide a thorough understanding of various embodiments of the present technology. In other instances, well-known structures, materials, operations, and/or systems often associated with nuclear reactors, power plant systems, integrated energy systems, chemical production plants, industrial process plants, electrolysis systems, direct air capture (DAC) plants, oil refineries, and the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology.

The accompanying Figures depict embodiments of the present technology and are not intended to limit its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

Each of the references cited herein is incorporated herein by reference in its entirety. However, to the extent any materials incorporated herein by reference conflict with the present disclosure, the present disclosure controls. The headings provided herein are for convenience only and should not be construed as limiting the subject matter disclosed.

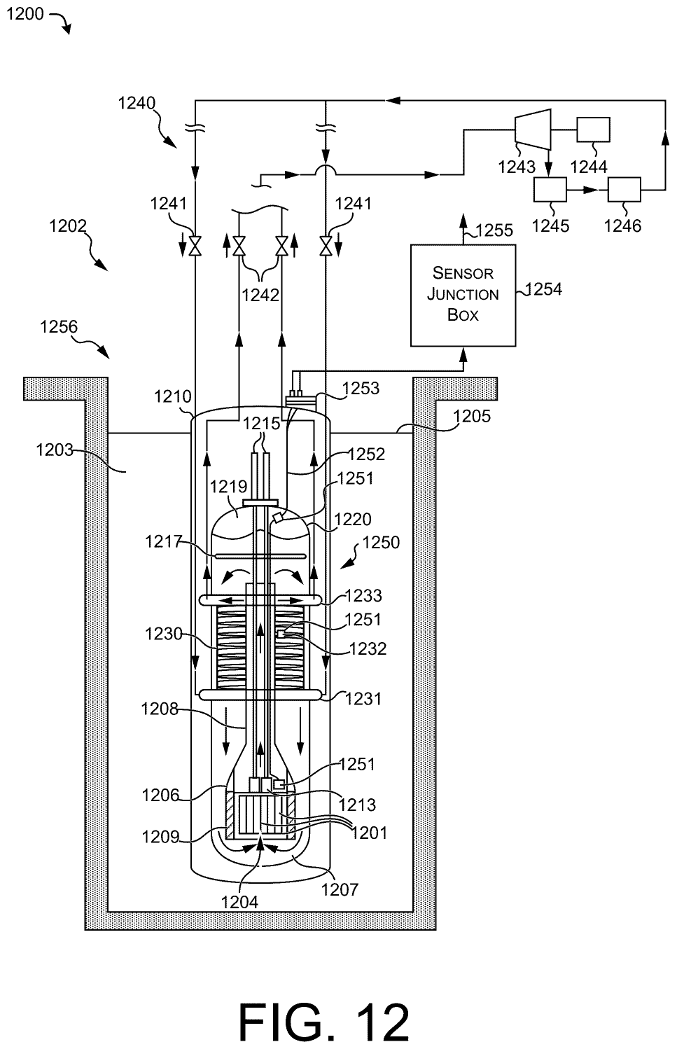

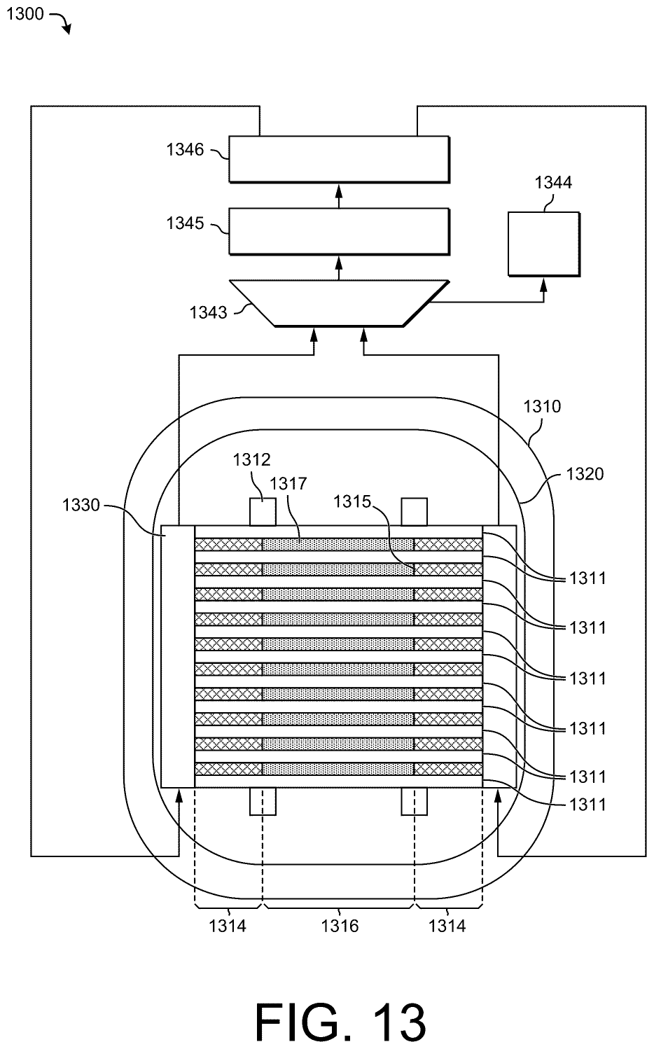

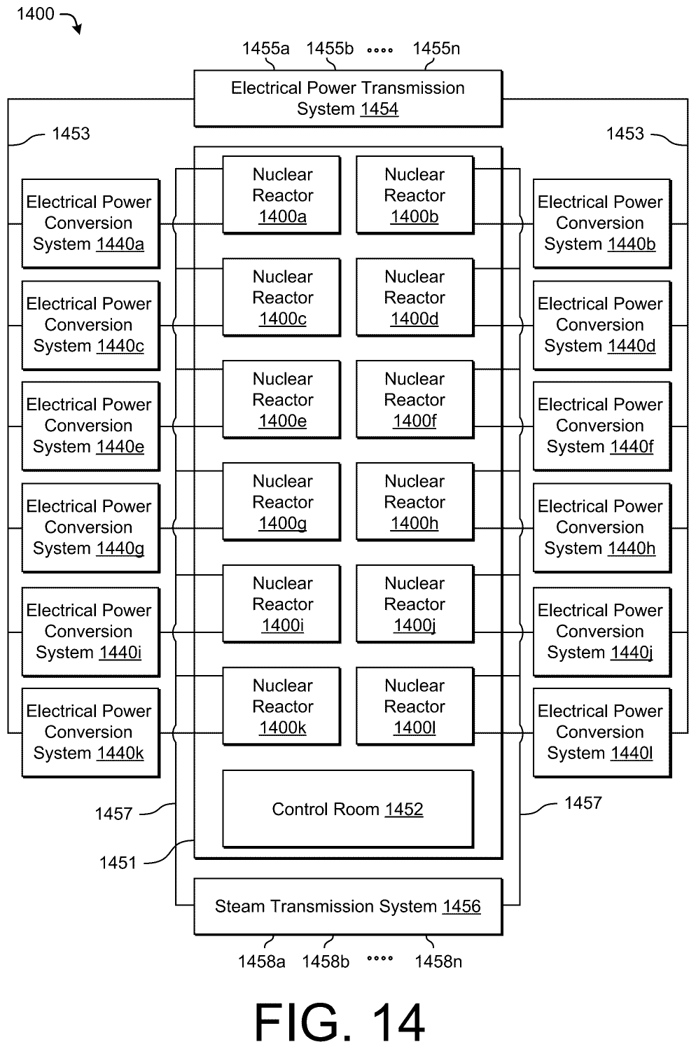

FIG. 1 schematically illustrates a representation of an integrated energy system (IES) 100 that includes a SMR system integrated with a Thermal Decomposition and Methanol (CH₃OH) production system. The IES 100 may include a power plant system 102, a power grid 104, a desalination system 106, a brine processing system 108, a Sodium Formate (HCOONa) production system 110, a Thermal Decomposition system 112, a Methanol (CH₃OH) production system 114, and Methanol (CH₃OH) Storage 116. In embodiments the power plant system 102 may include the power plant system 1450 of FIG. 14, in accordance with additional embodiments of the present technology. The power plant system 102 may include a nuclear power module (NPM) including one or more light water nuclear reactor (LWR), one or more small modular nuclear reactor (SMR), and any reactor 1200 of FIG. 12, reactor system 1300 of FIG. 13, and system of nuclear reactors 1400 of FIG. 14.

In the illustrated embodiment, the power plant system 102 can be configured to provide electrical power directly to the power grid 104. In embodiments, the power plant system 102 may produce and deliver electrical power to the power grid 104 during peak times or anytime that there is a demand for energy production. For example, when consumer energy demand imposes a high energy demand on the power grid 104 (“peak times”), the power plant system 102 may be configured to produce and provide the energy necessary for the power grid 104 to meet the high consumer demand during peak times. In embodiments, the power plant system 102 may be configured to provide energy directly to the power grid 104 as required to meet energy demand due to factors other than increased energy demand during peak times (e.g., an energy producing plant that provides energy to the power grid 104 may be offline and unable to provide energy, which creates an increased energy production demand without an increased demand for consumer electrical power).

In the illustrated embodiment, the power plant system 102 may be configured to provide steam and power to the desalination system 106. The desalination system 106 may include one or more water treatment processes, such as Reverse Osmosis (RO), distillation, and filtration. The desalination system 106 may receive supply water from a water source. In embodiments, the water source includes a natural body of water, such as an ocean, a sea, or a lake, a storage tank, an industrial process, and a separate water treatment facility. In embodiments, the desalination system 106 may be configured to utilize the steam and power from the power plant system 102 to convert the supply water into a concentrated NaCl solution (“brine”) and clean water. Supply water can include saline water. In embodiments, the supply water may have a dissolved salt concentration of 0.05% to 50%. For example, sea water from the world's oceans has a salt concentration of about 3.5%, and saltwater lakes and seas around the world have salt concentrations from 0.59% to 50%. In embodiments, the supply water includes salts such as Sodium Chloride (NaCl).

In embodiments, the brine and the clean water may be directed into the brine processing system 108. The brine processing system 108 may be configured to convert brine into Sodium Hydroxide (NaOH), Hydrogen (H₂), and Chlorine (Cl₂). The brine processing system 108 can be an electrolysis process, such as a chlor-alkali process. In embodiments, the brine processing system 108 may include an ion-selective membrane configured to allow Sodium ions (Na+) to flow freely across the membrane, while Chloride ions (Cl−) and Hydroxide ions (OH−) are prevented from migrating across the membrane. The brine processing system 108 may include an anode and a cathode. At the anode, Chloride ions (Cl−) from the brine solution are oxidized to form Chlorine (Cl₂) gas. At the cathode, water (H₂O) is reduced to Hydroxide ions (OH−) and Hydrogen (H₂) gas, releasing Hydroxide ions (OH−) into the solution. Sodium ions (Na+) from the brine solution flow across the membrane toward the cathode and combine with Hydroxide ions (OH−) to produce a Sodium Hydroxide (NaOH) solution. The Sodium Hydroxide (NaOH) solution may be removed as a product from the brine processing system 108. In embodiments, the Sodium Hydroxide (NaOH) may be converted to a solid form through a drying process, such as a thermal vacuum dehydration chamber.

In embodiments, the brine processing system 108 can reduce the Sodium Chloride (NaCl) concentration of brine. A processed water outlet stream from the brine processing system 108 may, for example, be reduced to a benign saline water concentration of Sodium Chloride (NaCl) (e.g., 3.5%). The processed water outlet stream may be further processed in downstream brine processing systems or fed back to the desalination system 106. In embodiments, the Chlorine (Cl₂) gas and Hydrogen (H₂) gas generated in the brine processing system 108 may be removed as a product to be stored, sold, or used in further resource production, such as in a Hydrochloric Acid production process. In embodiments, the desalination system, and the brine processing system 108 are configured to receive power from the power plant system 102.

In embodiments, the Sodium Hydroxide (NaOH) may be directed from the brine processing system 108 to the Sodium Formate (HCOONa) production system 110. The Sodium Formate (HCOONa) production system 110 may be configured to convert the Sodium Hydroxide (NaOH) from the brine processing system 108 into Sodium Formate (HCOONa). In embodiments, the Sodium Hydroxide (NaOH) solution can be used to capture carbon dioxide from the ambient air via a Direct Air Capture (DAC) process. The aqueous Sodium Hydroxide (NaOH) solution can chemically react with Carbon Dioxide (CO₂), for example from ambient or a carbon dioxide emission source, to form Sodium Carbonate (Na₂CO₃) and Sodium Bicarbonate (NaHCO₃). The Sodium Carbonate (Na₂CO₃) and Sodium Bicarbonate (NaHCO₃) may then be reacted with a carboxylic acid, such as Formic Acid, to generate Sodium Formate (HCOONa). In embodiments, Sodium Formate (HCOONa) can be produced by neutralizing Formic Acid (HCOOH) with the Sodium Hydroxide (NaOH) solution. In embodiments, the Sodium Hydroxide (NaOH) solution from the brine processing system 108 can be converted to a solid form through a drying process, such as a thermal vacuum dehydration chamber. Carbon Monoxide (CO) can then be absorbed by the solid Sodium Hydroxide (NaOH) under pressure to produce Sodium Formate (HCOONa), for example at a temperature of 130° C. and a pressure between 6-8 bar. In embodiments, Sodium Formate (HCOONa) may be produced in a large-scale inexpensively from Formic Acid (HCOOH) via carbonylation of Methanol (CH₃OH) followed by adding water to the resulting Methyl Formate (HCOOCH₃).

The Sodium Formate (HCOONa) from the Sodium Formate (HCOONa) production system 110 may be directed into the Thermal Decomposition system 114. In embodiments, the Thermal Decomposition system 114 may be configured to receive steam and/or electricity from the power plant system 102 to thermally decompose Sodium Formate (HCOONa) from the Sodium Formate (HCOONa) production system 110. The steam and/or electricity from the power plant system 102 can be used to heat the Thermal Decomposition system 112. The steam, for example, can provide thermal energy to heat the Thermal Decomposition system 112 indirectly through a jacketed reaction chamber or heat exchanger. In embodiments the steam can provide thermal energy to heat the Thermal Decomposition system 112 directly to an interior of a reaction chamber. The Thermal Decomposition system 112 can produce Hydrogen (H₂), Carbon Monoxide (CO), and Carbon Dioxide (CO₂) from an alkali metal formate, such as Sodium Formate (HCOONa).

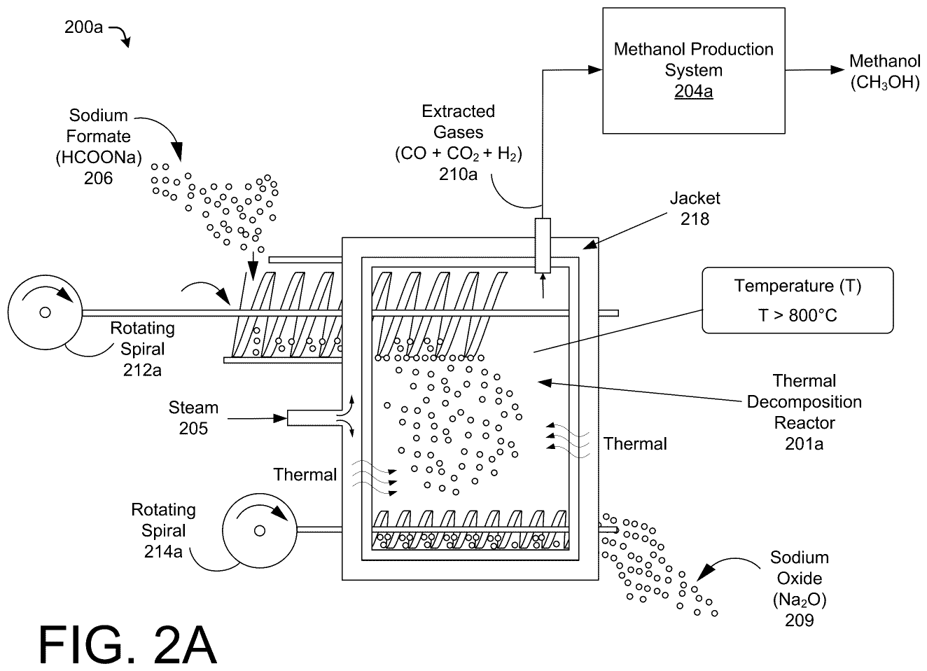

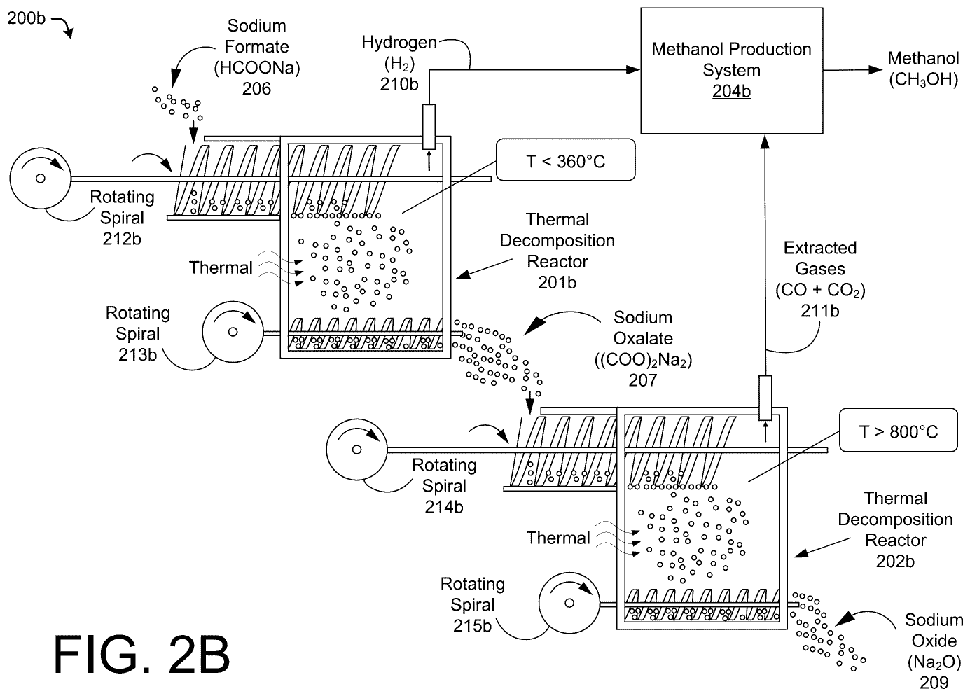

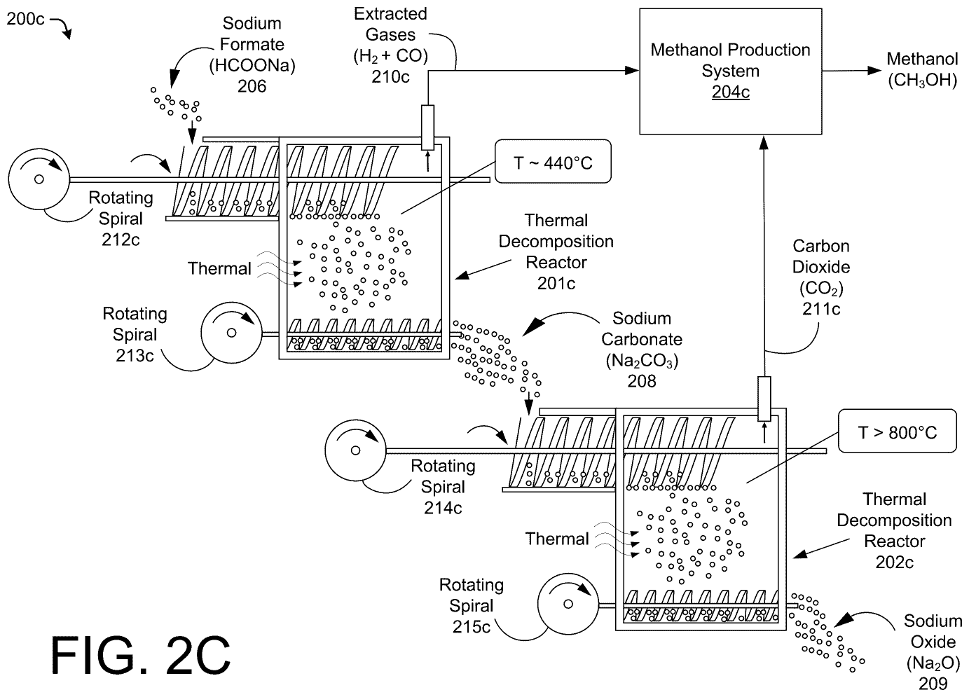

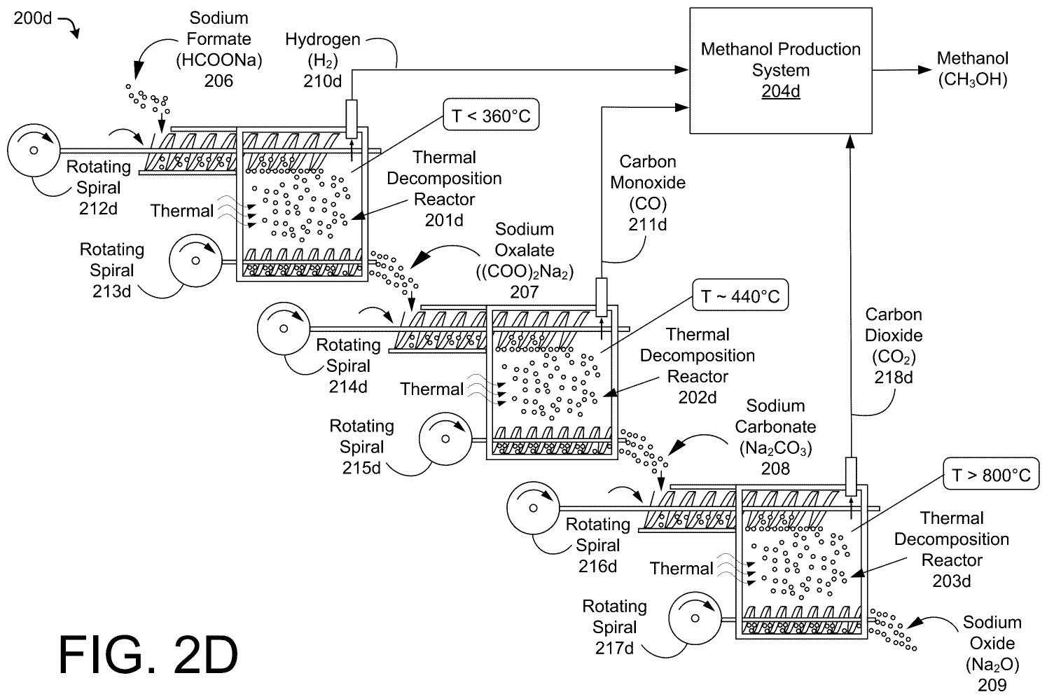

In embodiments, the Thermal Decomposition system 112 may include one or more thermal decomposition reaction chambers. In embodiments, Hydrogen (H₂) can be formed in a formate to oxalate coupling reaction (FOCR) within a thermal decomposition reaction chamber. For example, Sodium Formate (HCOONa) from the Sodium Formate (HCOONa) production system 110 can be heated to form Hydrogen (H₂) and Sodium Oxalate ((COO)₂Na₂). In embodiments, the Sodium Oxalate ((COO)₂Na₂) can remain in same the thermal decomposition reaction chamber or can be fed to a separate thermal decomposition reaction chamber to undergo additional heating and thermal decomposition to form Sodium Carbonate (Na₂CO₃) and Carbon Monoxide (CO). In embodiments, the Sodium Carbonate (Na₂CO₃) can remain in same the thermal decomposition reaction chamber or can be fed to a separate thermal decomposition reaction chamber to undergo additional heating and thermal decomposition to form Sodium Oxide (Na₂O) and Carbon Dioxide (CO₂). The Hydrogen (H₂), Carbon Monoxide (CO), and Carbon Dioxide (CO₂) formed in the Thermal Decomposition system 112 can be fed to the Methanol (CH₃OH) production system 114. In embodiments, Sodium Formate (HCOONa) decomposition into Sodium Oxalate ((COO)₂Na₂) and Hydrogen (H₂), Sodium Carbonate (Na₂CO₃) and Carbon Monoxide (CO), and Sodium Oxide (Na₂O) and Carbon Dioxide (CO₂) may be dependent on temperature and/or rate of heating. In embodiments, the Thermal Decomposition system 112 can be configured with any number of Thermal Decomposition reaction chambers optimized to a temperature and/or residence time that will maximize the yield and/or efficiency of producing the desired thermal decomposition product(s). See, for example, FIGS. 2A-D.

In embodiments, the IES 100 may include a Methanol (CH₃OH) production system 114. The Methanol (CH₃OH) production system 114 may include a high selectivity Copper (Cu) and zinc oxide (ZnO) based catalyst (Cu/ZnO). The reaction of Carbon Dioxide (CO₂) and Hydrogen (H₂) generate water which accelerates the deactivation of the Cu/ZnO catalyst. The Carbon Monoxide (CO) within the Methanol (CH₃OH) production system 114 removes water from the reaction of Carbon Dioxide (CO₂) and Hydrogen (H₂) via the Water-Gas-Shift (WGS) reaction. Methanol (CH₃OH) produced in the Methanol (CH₃OH) production system 114 may be stored or sold. In embodiments, the Methanol (CH₃OH) from the Methanol (CH₃OH) production system 114 may be directed to the Methanol (CH₃OH) storage 116. In embodiments, the Methanol (CH₃OH) production system 114 is configured to receive power and/or steam from the power plant system 102.

In embodiments, intermediate products within the IES 100 may be removed from the IES and/or recycled within the IES to supply inputs for one or more processes within the IES. For example, one or more intermediate products such as clean water, Sodium Hydroxide (NaOH), Hydrogen (H₂), Chlorine (Cl₂), Carbon Monoxide (CO), Carbon Dioxide (CO₂), Sodium Formate (NaHCOO), Sodium Oxalate (COO)₂Na₂), Sodium Carbonate (Na₂CO₃), Sodium Oxide (Na₂O), and Methanol (CH₃OH) may be removed from the IES to be stored for later use, to be sold, or to be recycled within the IES 100 to optimize yield, process efficiency, cost effectiveness, or energy utilization.

In embodiments, the IES 100 may include a control system. In representative embodiments, the IES 100 has a steady source of electric power from the power plant system 102 and supply water, available all the time at the desired quantity. The optimal production rate of Methanol (CH₃OH) requires supplying the proper amount of Sodium Formate (HCOONa) to the Thermal Decomposition system 112 and supplying the proper ratio of Hydrogen (H₂), Carbon Dioxide (CO₂), and Carbon Monoxide (CO) to the Methanol production system 114. A closed loop control system can be implemented to optimize Sodium Formate (HCOONa) and Methanol (CH₃OH) production, process efficiency, cost effectiveness, and/or energy utilization. The closed loop control system may include a microcontroller or computer that measures the concentration of process streams and adjusts the production of Sodium Formate (HCOONa) and Methanol (CH₃OH) accordingly by adjusting the power applied from the power plant system 102 to the IES 100. The power saved from the above process can, for example, be sourced to the power grid 104 or sourced to the desalination system 106 to produce a larger volume of clean water.

It is understood that the IES 100 may be configured such that the power plant system 102 may simultaneously produce electrical power directly to the power grid 104 and produce Methanol (CH₃OH) via the desalination system 106, the brine processing system 108, the Sodium Formate (HCOONa) production system 110, the Thermal Decomposition system 112, and the Methanol (CH₃OH) production system 114. In embodiments, the power plant system 102 can be a permanent or temporary installation built at or near (e.g., roughly 1 km from) the location of the power grid 104, the desalination system 106, the brine processing system 108, the Sodium Formate (HCOONa) production system 110, the Thermal Decomposition system 112, the Methanol (CH₃OH) production system 114, and the Methanol (CH₃OH) storage 116. In embodiments, the power plant system 102 can be a mobile or partially mobile system that is moved to and assembled at or near the location of the power grid 104 the desalination system 106, the brine processing system 108, the Sodium Formate (HCOONa) production system 110, the Thermal Decomposition system 112, the Methanol (CH₃OH) production system 114, and the Methanol (CH₃OH) storage 116. More generally, the power plant system 102 can be local (e.g., positioned at or near) to any one or more of the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports.

FIG. 2A schematically illustrates a representative schematic diagram of a Thermal Decomposition system 200a (“system 200a”) during steady state operations. In embodiments, the system 200a may include a Thermal Decomposition reactor 201a and a Methanol production system 204a. The Thermal Decomposition reactor 201a may be configured to receive Sodium Formate (HCOONa) 206 and to produce Sodium Oxide (Na₂O) 209 and extracted gases 210a. The Sodium Formate (HCOONa) 206 may be directed, via the rotating spiral 212a (e.g., first rotating spiral), into an upper portion of the Thermal Decomposition reactor 201a. The Thermal Decomposition reactor 201a may have an internal temperature of >800° C. The Thermal Decomposition reactor 201a may be configured to receive thermal energy to maintain the internal temperature, for example, from a power plant such as power plant system 102. The thermal energy may include indirect heat transfer from steam 205 supplied by the power plant. For example, the Thermal Decomposition reactor 201a may include a jacket 218 that receives the steam 205 and transfers heat through the walls of the jacket 218 such that no steam 205 comes directly into contact with the interior of the Thermal Decomposition reactor 201a. In embodiments, additional thermal energy may be added to the steam 205 supplied by the power plant to indirectly heat the Thermal Decomposition reactor 201a to the desired temperature, for example, through compression and/or heating of the steam 205 in auxiliary compressors and/or heaters. In embodiments, auxiliary compressors and/or heaters may be powered by electrical energy from the power plant. In embodiments, the thermal energy may include heat provided to the Thermal Decomposition reactor 201a by electrical heaters powered by electrical energy from the power plant. In embodiments, the Thermal Decomposition reactor 201a may receive any combination of steam and/or electrical heating from the power plant to maintain the desired internal temperature.

Within the Thermal Decomposition reactor 201a, the Sodium Formate (HCOONa) 206 may undergo the reactions in Equations 4-6 at a temperature of >800° C. to produce Sodium Oxide (Na₂O) 209 and the extracted gases 210a (i.e. Hydrogen (H₂), Carbon Monoxide (CO), and Carbon Dioxide (CO₂). The thermal decomposition processes are:
(1) Sodium Formate (HCOONa) 206 first undergoes the thermal decomposition reaction in Equation 4 to produce Sodium Oxalate ((COO)₂Na₂) and Hydrogen (H₂):

2HCOONa→(COO)₂Na₂+H₂ (4)

(2) The Sodium Oxalate ((COO)₂Na₂) then undergoes the thermal decomposition reaction in Equation 5 to produce Sodium Carbonate (Na₂CO₃) and Carbon Monoxide (CO):

(COO)₂Na₂→Na₂CO₃+CO (5)
(3) The Sodium Carbonate (Na₂CO₃) then undergoes the thermal decomposition reaction in Equation 6 to produce Sodium Oxide (Na₂O) 209 and Carbon Dioxide (CO₂):

Na₂CO₃→Na₂O+CO₂ (6)

In embodiments, Sodium Formate (HCOONa) 206 may be in a solid state and ground into fine powder via the rotating spiral 212a such that the Sodium Formate (HCOONa) 206 is disintegrated within the chamber. In various cases, the rotating spiral 212a may be utilized to convert the Sodium Formate (HCOONa) 206 between particles of different sizes. For example, the rotating spiral 212a may be utilized to convert the Sodium Formate (HCOONa) 206 from relatively coarser (e.g., larger) particles to relatively finer (e.g., smaller) particles. The rotating spiral 212a may be utilized to assist in the conversion of the Sodium Formate (HCOONa) 206 to the Sodium Oxide (Na₂O) 209. In embodiments, the rotating spiral 212a may be utilized to assist in the conversion of the Sodium Formate (HCOONa) 206 to Sodium Oxalate ((COO)₂Na₂), Sodium Carbonate (Na₂CO₃), and the Sodium Oxide (Na₂O) 209. The rotating spiral 212a may be utilized to maintain the temperature in the Thermal Decomposition reactor 201a by providing a means to feed the Sodium Formate (HCOONa) 206 into the upper portion of the Thermal Decomposition reactor 201a while minimizing the potential for heat loss. The rotating spiral 212a may be a metal rotating spiral (e.g., an auger), which may be located partially and/or fully in the first Thermal Decomposition reactor 201a. In embodiments, the rotating spiral 212a may be operated by a control system utilized to control any portion of the system 200a to rotate (e.g., spin) at one or more predetermined, and/or dynamically determined (e.g., in real-time), speeds during any operation of the system 200a.

In embodiments, the Sodium Formate (HCOONa) 206, may receive thermal energy as a result of the temperature inside the Thermal Decomposition reactor 201a. The internal temperature of the Thermal Decomposition reactor 201a may cause the Sodium Formate (HCOONa) 206 to rapidly decompose into the extracted gases 210a and the Sodium Oxide (Na₂O) 209. For example, the Hydrogen (H₂) may be produced instantaneously following the decomposition of the Sodium Formate (HCOONa) 206 into the Sodium Oxalate ((COO)₂Na₂), the Carbon Monoxide (CO) may be produced instantaneously following the decomposition of the Sodium Oxalate (COO)₂Na₂) into the Sodium Carbonate (Na₂CO₃), and the Carbon Dioxide (CO₂) may be produced instantaneously following the decomposition of the Sodium Carbonate (Na₂CO₃) into the Sodium Oxide (Na₂O) 209. In the embodiment, the resulting Sodium Oxide (Na₂O) 209 sinks to the bottom of the Thermal Decomposition reactor 201a while still being thermally hot. The rotating spiral 214a (e.g., second rotating spiral) may transfer the thermally hot Sodium Oxide (Na₂O) 209 from the bottom of the Thermal Decomposition reactor 201a to outside the Thermal Decomposition reactor 201a for collection and/or additional industrial use and/or processing.

In embodiments, the extracted gases 210a may be removed from the Thermal Decomposition reactor 201a for collection and/or additional industrial use and/or processing. For example, a pressure swing adsorption system may be used to separate and purify the Hydrogen (H₂), Carbon Monoxide (CO), and Carbon Dioxide (CO₂) extracted from the Thermal Decomposition reactor 201a for future use and/or processing. In embodiments, a portion of one or more of the extracted gases 210a may be directed for collection and/or additional industrial use and/or processing. For example, the Hydrogen (H₂) may be directed into a Hydrogen Fuel Cell and used for producing electricity.

In embodiments, the extracted gases 210a may be fed to the Methanol production system 204a. In embodiments, the Methanol (CH₃OH) synthesis reactions in Equations 1-3 take place within the Methanol production system 204a over a Cu/ZnO catalyst at a temperature between 200-300° C. and at a pressure between 50-100 bar. Even though Methanol (CH₃OH) can be produced by directly hydrogenating pure Carbon Dioxide (CO₂) with Hydrogen (H₂) via Equations 1 & 2 with conventional Cu/ZnO-based catalysts, the reaction rates will be terminated shortly after initiation due to the formation of water that leads to kinetic inhibition and the accelerated deactivation of the Cu/ZnO catalysts. Therefore, the presence of Carbon Monoxide (CO) in the reaction is extremely important to continue the WGS reaction in order to maintain the synthesis at low temperature and high pressure without the deactivation of the catalyst.

In embodiments, Hydrogen (H₂), Carbon Monoxide (CO), and Carbon Dioxide (CO₂) are fed into the Methanol production system 204a from the Thermal Decomposition reactor 201a. In embodiments, the extracted gases 210a are fed through a pressure swing adsorption system to separate and purify the extracted Hydrogen (H₂), Carbon Monoxide (CO), and Carbon Dioxide (CO₂) prior to entering the Methanol production system 204a. In embodiments, separated and purified Hydrogen (H₂), Carbon Monoxide (CO), and Carbon Dioxide (CO₂) may be fed into the Methanol production system 204a individually at a rate that may optimize the production of Methanol (CH₃OH). The produced Methanol (CH₃OH) may be stored or sold. In embodiments, the Methanol production system 204a is configured to receive power from a power plant, such as the power plant system 102.

FIG. 2B schematically illustrates a representative schematic diagram of a Thermal Decomposition system 200b (“system 200b”) during steady state operations. In embodiments, the system 200b may include a Thermal Decomposition reactor 201b, a Thermal Decomposition reactor 202b, and a Methanol production system 204b. The Thermal Decomposition reactors 201b and 202b may be configured to receive thermal energy to maintain the desired internal temperature, for example, from a power plant such as power plant system 102. The thermal energy may include indirect heat transfer from steam supplied by the power plant. For example, the Thermal Decomposition reactor 201b and 202b may each include a jacket that receives the steam and transfers heat through the walls of the jacket such that no steam comes directly into contact with the interior of the reactor. In embodiments, additional thermal energy may be added to steam supplied by the power plant to indirectly heat the Thermal Decomposition reactor 201b and 202b to the desired temperature, for example, through compression and/or heating of the steam in auxiliary compressors and/or heaters. In embodiments, auxiliary compressors and/or heaters may be powered by electrical energy from the power plant. In embodiments, the thermal energy may include heat provided to Thermal Decomposition reactor 201b and 202b by electrical heaters powered by electrical energy from the power plant. In embodiments, the Thermal Decomposition reactor 201b and 202b may receive any combination of steam and/or electrical heating from the power plant to maintain the desired internal temperature.

The Thermal Decomposition reactor 201b may be configured to receive Sodium Formate (HCOONa) 206 and to produce Sodium Oxalate ((COO)₂Na₂) 207 and Hydrogen (H₂) 210b. The Sodium Formate (HCOONa) 206 may be directed, via the rotating spiral 212b (e.g., first rotating spiral), into an upper portion of the Thermal Decomposition reactor 201b. The Thermal Decomposition reactor 201b may have an internal temperature of <450° C., <400° C., or <360° C. Within the Thermal Decomposition reactor 201b, the Sodium Formate (HCOONa) 206 may undergo the reaction in Equation 4 to produce Sodium Oxalate ((COO)₂Na₂) 207 and Hydrogen (H₂) 210b:

2HCOONa→(COO)₂Na₂+H₂ (4)

In embodiments, Sodium Formate (HCOONa) 206 may be in a solid state and ground into fine powder via the rotating spiral 212b such that the Sodium Formate (HCOONa) 206 is disintegrated within the chamber. In various cases, the rotating spiral 212b may be utilized to convert the Sodium Formate (HCOONa) 206 between particles of different sizes. For example, the rotating spiral 212b may be utilized to convert the Sodium Formate (HCOONa) 206 from relatively coarser (e.g., larger) particles to relatively finer (e.g., smaller) particles. The rotating spiral 212b may be utilized to assist in the conversion of the Sodium Formate (HCOONa) 206 to the Sodium Oxalate ((COO)₂Na₂) 207. The rotating spiral 212b may be utilized to maintain the temperature in the Thermal Decomposition reactor 201b by providing a means to feed the Sodium Formate (HCOONa) 206 into the upper portion of the Thermal Decomposition reactor 201b while minimizing the potential for heat loss.

In embodiments, the Sodium Formate (HCOONa) 206, may receive thermal energy as a result of the temperature inside the Thermal Decomposition reactor 201b. The internal temperature of the Thermal Decomposition reactor 201b may cause the Sodium Formate (HCOONa) 206 to rapidly decompose into Hydrogen (H₂) 210b and the Sodium Oxalate ((COO)₂Na₂) 207. In the embodiment, the resulting Sodium Oxalate ((COO)₂Na₂) 207 sinks to the bottom of the Thermal Decomposition reactor 201b while still being thermally hot. The rotating spiral 213b (e.g., second rotating spiral) may transfer the thermally hot Sodium Oxalate ((COO)₂Na₂) 207 from the bottom of the Thermal Decomposition reactor 201b to outside the Thermal Decomposition reactor 201b for collection and/or additional industrial use and/or processing. For example, the Sodium Oxalate ((COO)₂Na₂) 207 from the bottom of the Thermal Decomposition reactor 201b may be directed, via the rotating spiral 214b (e.g., third rotating spiral), into an upper portion of the Thermal Decomposition reactor 202b. In embodiments, the rotating spiral 213b and the rotating spiral 214b may be connected via a thermally insulated conduit. The rotating spiral 213b and the rotating spiral 214b may be utilized to maintain the temperature in the Thermal Decomposition reactor 201b and the Thermal Decomposition reactor 202b by providing a means to feed the Sodium Oxalate ((COO)₂Na₂) 207 into the upper portion of the Thermal Decomposition reactor 202b while minimizing the potential for heat loss. In embodiments, the rotating spiral 213b and the rotating spiral 214b may be the same rotating spiral.

The Thermal Decomposition reactor 202b may be configured to receive Sodium Oxalate ((COO)₂Na₂) 207 and to produce Sodium Oxide (Na₂O) 209 and extracted gases 211b. The Thermal Decomposition reactor 202b may have an internal temperature of >800° C. Within the Thermal Decomposition reactor 202b, the Sodium Oxalate ((COO)₂Na₂) 207 undergoes the thermal decomposition reactions in Equations 5 and 6 to produce Sodium Oxide (Na₂O) and the extracted gases 211b (i.e., Carbon Monoxide (CO), and Carbon Dioxide (CO₂)). The thermal decomposition processes are:
(1) Sodium Oxalate ((COO)₂Na₂) 207 first undergoes the decomposition reaction in Equation 5 to produce Sodium Carbonate (Na₂CO₃) and Carbon Monoxide (CO):

(COO)₂Na₂→Na₂CO₃+CO (5)
(2) The produced Sodium Carbonate (Na₂CO₃) then undergoes the reaction in Equation 6 to produce Sodium Oxide (Na₂O) 209 and Carbon Dioxide (CO₂):

Na₂CO₃→Na₂O+CO₂ (6)

In embodiments, Sodium Oxalate (COO)₂Na₂) 207 may be in a solid state and ground into fine powder via the rotating spiral 214b such that the Sodium Oxalate ((COO)₂Na₂) 207 is disintegrated within the chamber. In various cases, the rotating spiral 214b may be utilized to convert the Sodium Oxalate ((COO)₂Na₂) 207 between particles of different sizes. For example, the rotating spiral 214b may be utilized to convert the Sodium Oxalate ((COO)₂Na₂) 207 from relatively coarser (e.g., larger) particles to relatively finer (e.g., smaller) particles. The rotating spiral 214b may be utilized to assist in the conversion of the Sodium Oxalate ((COO)₂Na₂) 207 to the Sodium Oxide (Na₂O) 209. In embodiments, the rotating spiral 214b may be utilized to assist in the conversion of the Sodium Oxalate ((COO)₂Na₂) 207 to Sodium Carbonate (Na₂CO₃) and the Sodium Oxide (Na₂O) 209.

In embodiments, the Sodium Oxalate ((COO)₂Na₂) 207, may receive thermal energy as a result of the temperature inside the Thermal Decomposition reactor 202b. The internal temperature of the Thermal Decomposition reactor 202b may cause the Sodium Oxalate ((COO)₂Na₂) 207 to rapidly decompose into the extracted gases 211b and the Sodium Oxide (Na₂O) 209. For example, the Carbon Monoxide (CO) may be produced instantaneously following the decomposition of the Sodium Oxalate (COO)₂Na₂) 207 into the Sodium Carbonate (Na₂CO₃), and the Carbon Dioxide (CO₂) may be produced instantaneously following the decomposition of the Sodium Carbonate (Na₂CO₃) into the Sodium Oxide (Na₂O) 209. In the embodiment, the resulting Sodium Oxide (Na₂O) 209 sinks to the bottom of the Thermal Decomposition reactor 202b while still being thermally hot. The rotating spiral 215b (e.g., fourth rotating spiral) may transfer the thermally hot Sodium Oxide (Na₂O) 209 from the bottom of the Thermal Decomposition reactor 202b to outside the Thermal Decomposition reactor 202b for collection and/or additional industrial use and/or processing. The rotating spirals 212b, 213b, 214b, and 215b may each be a metal rotating spiral (e.g., an auger), which may be located partially and/or fully in the Thermal Decomposition reactors. In embodiments, the rotating spirals 212b, 213b, 214b, and 215b may be operated by a control system utilized to control any portion of the system 200b to rotate (e.g., spin) at one or more predetermined, and/or dynamically determined (e.g., in real-time), speeds during any operation of the system 200b.

In embodiments, the Hydrogen (H₂) 210b and the extracted gases 211b may be removed from the Thermal Decomposition reactor 201b and the Thermal Decomposition reactor 202b, respectively, for collection and/or additional industrial use and/or processing. For example, a pressure swing adsorption system may be used to separate and purify the Hydrogen (H₂), Carbon Monoxide (CO), and Carbon Dioxide (CO₂) extracted from the Thermal Decomposition reactors for future use and/or processing. In embodiments, a portion of one or more of the Hydrogen (H₂), Carbon Monoxide (CO), and Carbon Dioxide (CO₂) extracted from the Thermal Decomposition reactors may be directed for collection and/or additional industrial use and/or processing. For example, the Hydrogen (H₂) may be directed into a portion of an electrochemical device (e.g., a hydrogen fuel cell anode) and used for producing electricity.

In embodiments, the Hydrogen (H₂) and extracted gases 211b may be fed to the Methanol production system 204b. In embodiments, the Methanol (CH₃OH) synthesis reactions in Equations 1-3 take place within the Methanol production system 204b over a Cu/ZnO catalyst at a temperature between 200-300° C. and at a pressure between 50-100 bar. In embodiments, Hydrogen (H₂) is fed into the Methanol production system 204b from the Thermal Decomposition reactor 201b, and Carbon Monoxide (CO) and Carbon Dioxide (CO₂) are fed into the Methanol production system 204b from the Thermal Decomposition reactor 202b. In embodiments, the Hydrogen (H₂) 210b and the extracted gases 211b are fed through a pressure swing adsorption system to separate and purify the extracted Hydrogen (H₂), Carbon Monoxide (CO), and Carbon Dioxide (CO₂) prior to entering the Methanol production system 204b. In embodiments, separated and purified Hydrogen (H₂), Carbon Monoxide (CO), and Carbon Dioxide (CO₂) may be fed into the Methanol production system 204b individually at a rate that may optimize the production of Methanol (CH₃OH). The produced Methanol (CH₃OH) may be stored or sold. In embodiments, the Methanol production system 204b is configured to receive power from a power plant, such as the power plant system 102.

FIG. 2C schematically illustrates a representative schematic diagram of a Thermal Decomposition system 200c (“system 200c”) during steady state operations. In embodiments, the system 200c may include a Thermal Decomposition reactor 201c, a Thermal Decomposition reactor, 202c and a Methanol production system 204c. The Thermal Decomposition reactors 201c and 202c may be configured to receive thermal energy to maintain the desired internal temperature, for example, from a power plant such as power plant system 102. The thermal energy may include indirect heat transfer from steam supplied by the power plant. For example, the Thermal Decomposition reactors 201c and 202c may each include a jacket that receives the steam and transfers heat through the walls of the jacket such that no steam comes directly into contact with the interior of the reactor. In embodiments, additional thermal energy may be added to steam supplied by the power plant to indirectly heat the Thermal Decomposition reactors 201c and 202c to the desired temperature, for example, through compression and/or heating of the steam in auxiliary compressors and/or heaters. In embodiments, auxiliary compressors and/or heaters may be powered by electrical energy from the power plant. In embodiments, the thermal energy may include heat provided to Thermal Decomposition reactors 201c and 202c by electrical heaters powered by electrical energy from the power plant. In embodiments, the Thermal Decomposition reactors 201c and 202c may receive any combination of steam and/or electrical heating from the power plant to maintain the desired internal temperature.

The Thermal Decomposition reactor 201c may be configured to receive Sodium Formate (HCOONa) 206 and to produce Sodium Carbonate (Na₂CO₃) 208 and extracted gases 210c. The Sodium Formate (HCOONa) 206 may be directed, via the rotating spiral 212c (e.g., first rotating spiral), into an upper portion of the Thermal Decomposition reactor 201c. The Thermal Decomposition reactor 201c may have an internal temperature of about 400-450° C., for example about 440° C. Within the Thermal Decomposition reactor 201c, the Sodium Formate (HCOONa) 206 may undergo the reactions in Equations 4 and 5 to produce Sodium Carbonate (Na₂CO₃) 208 and extracted gases 210c (i.e., Hydrogen (H₂) and Carbon Monoxide (CO).

The thermal decomposition processes are:
(1) Sodium Formate (HCOONa) 206 first undergoes the decomposition reaction in Equation 4 to produce Sodium Oxalate (COO)₂Na₂) and Hydrogen (H₂):

2HCOONa→(COO)₂Na₂+H₂ (4)
(2) Sodium Oxalate ((COO)₂Na₂) then undergoes the thermal decomposition reaction in Equation 5 to produce Sodium Carbonate (Na₂CO₃) 208 and Carbon Monoxide (CO):

(COO)₂Na₂→Na₂CO₃+CO (5)

In embodiments, Sodium Formate (HCOONa) 206 may be in a solid state and ground into fine powder via the rotating spiral 212c such that the Sodium Formate (HCOONa) 206 is disintegrated within the chamber. In various cases, the rotating spiral 212c may be utilized to convert the Sodium Formate (HCOONa) 206 between particles of different sizes. For example, the rotating spiral 212c may be utilized to convert the Sodium Formate (HCOONa) 206 from relatively coarser (e.g., larger) particles to relatively finer (e.g., smaller) particles. The rotating spiral 212c may be utilized to assist in the conversion of the Sodium Formate (HCOONa) 206 to the Sodium Carbonate (Na₂CO₃) 208. The rotating spiral 212c may be utilized to maintain the temperature in the Thermal Decomposition reactor 201c by providing a means to feed the Sodium Formate (HCOONa) 206 into the upper portion of the Thermal Decomposition reactor 201c while minimizing the potential for heat loss.

In embodiments, the Sodium Formate (HCOONa) 206, may receive thermal energy as a result of the temperature inside the Thermal Decomposition reactor 201c. The internal temperature of the Thermal Decomposition reactor 201c may cause the Sodium Formate (HCOONa) 206 to rapidly decompose into extracted gases 210c (i.e., Hydrogen (H₂) and Carbon Monoxide (CO)) and the Sodium Carbonate (Na₂CO₃) 208. For example, the Hydrogen (H₂) may be produced instantaneously following the decomposition of the Sodium Formate (HCOONa) 206 into the Sodium Oxalate ((COO)₂Na₂) and the Carbon Monoxide (CO) may be produced instantaneously following the decomposition of the Sodium Oxalate ((COO)₂Na₂) into the Sodium Carbonate (Na₂CO₃) 208. In the embodiment, the resulting Sodium Carbonate (Na₂CO₃) 208 sinks to the bottom of the Thermal Decomposition reactor 201c while still being thermally hot. The rotating spiral 213c (e.g., second rotating spiral) may transfer the thermally hot Sodium Carbonate (Na₂CO₃) 208 from the bottom of the Thermal Decomposition reactor 201c to outside the Thermal Decomposition reactor 201c for collection and/or additional industrial use and/or processing. For example, the Sodium Carbonate (Na₂CO₃) 208 from the bottom of the Thermal Decomposition reactor 201c may be directed, via the rotating spiral 214c (e.g., third rotating spiral), into an upper portion of the Thermal Decomposition reactor 202c. In embodiments, the rotating spiral 213c and the rotating spiral 214c may be connected via a thermally insulated conduit. The rotating spiral 213c and the rotating spiral 214c may be utilized to maintain the temperature in the Thermal Decomposition reactor 201c and the Thermal Decomposition reactor 202c by providing a means to feed the Sodium Carbonate (Na₂CO₃) 208 into the upper portion of the Thermal Decomposition reactor 202c while minimizing the potential for heat loss. In embodiments, the rotating spiral 213c and the rotating spiral 214c may be the same rotating spiral.

The Thermal Decomposition reactor 202c may be configured to receive Sodium Carbonate (Na₂CO₃) 208 and to produce Sodium Oxide (Na₂O) 209 and Carbon Dioxide (CO₂) 211c. The Thermal Decomposition reactor 202c may have an internal temperature of >800° C. Within the Thermal Decomposition reactor 202c, the Sodium Carbonate (Na₂CO₃) 208 may undergo the reaction in Equation 6 to produce Sodium Oxide (Na₂O) 209 and the Carbon Dioxide (CO₂) 211c:

Na₂CO₃→Na₂O+CO₂ (6)

In embodiments, Sodium Carbonate (Na₂CO₃) 208 may be in a solid state and ground into fine powder via the rotating spiral 214c such that the Sodium Carbonate (Na₂CO₃) 208 is disintegrated within the chamber. In various cases, the rotating spiral 214c may be utilized to convert the Sodium Carbonate (Na₂CO₃) 208 between particles of different sizes. For example, the rotating spiral 214c may be utilized to convert the Sodium Carbonate (Na₂CO₃) 208 from relatively coarser (e.g., larger) particles to relatively finer (e.g., smaller) particles. The rotating spiral 214c may be utilized to assist in the conversion of the Sodium Carbonate (Na₂CO₃) 208 to the Sodium Oxide (Na₂O) 209.

In embodiments, the Sodium Carbonate (Na₂CO₃) 208 may receive thermal energy as a result of the temperature inside the Thermal Decomposition reactor 202c. The internal temperature of the Thermal Decomposition reactor 202c may cause the Sodium Carbonate (Na₂CO₃) 208 to rapidly decompose into the Carbon Dioxide (CO₂) 211c and the Sodium Oxide (Na₂O) 209. In the embodiment, the resulting Sodium Oxide (Na₂O) 209 sinks to the bottom of the Thermal Decomposition reactor 202c while still being thermally hot. The rotating spiral 215c (e.g., fourth rotating spiral) may transfer the thermally hot Sodium Oxide (Na₂O) 209 from the bottom of the Thermal Decomposition reactor 202c to outside the Thermal Decomposition reactor 202c for collection and/or additional industrial use and/or processing. The rotating spirals 212c, 213c, 214c, and 215c may each be a metal rotating spiral (e.g., an auger), which may be located partially and/or fully in the Thermal Decomposition reactors. In embodiments, the rotating spirals 212c, 213c, 214c, and 215c may be operated by a control system utilized to control any portion of the system 200c to rotate (e.g., spin) at one or more predetermined, and/or dynamically determined (e.g., in real-time), speeds during any operation of the system 200c.

In embodiments, the extracted gases 210c and the Carbon Dioxide (CO₂) 211c may be removed from the Thermal Decomposition reactor 201c and the Thermal Decomposition reactor 202c, respectively, for collection and/or additional industrial use and/or processing. For example, a pressure swing adsorption system may be used to separate and purify the Hydrogen (H₂), Carbon Monoxide (CO), and Carbon Dioxide (CO₂) extracted from the Thermal Decomposition reactors for future use and/or processing. In embodiments, a portion of one or more of the Hydrogen (H₂), Carbon Monoxide (CO), and Carbon Dioxide (CO₂) extracted from the Thermal Decomposition reactors may be directed for collection and/or additional industrial use and/or processing. For example, the Hydrogen (H₂) may be directed into a portion of an electrochemical device (e.g., a hydrogen fuel cell anode) and used for producing electricity.

In embodiments, the extracted gases 210c and the Carbon Dioxide (CO₂) 211c may be fed to the Methanol production system 204c. In embodiments, the Methanol (CH₃OH) synthesis reactions in Equations 1-3 take place within the Methanol production system 204c over a Cu/ZnO catalyst at a temperature between 200-300° C. and at a pressure between 50-100 bar. In embodiments, Hydrogen (H₂) and Carbon Monoxide (CO) are fed into the Methanol production system 204c from the Thermal Decomposition reactor 201c, and Carbon Dioxide (CO₂) is fed into the Methanol production system 204c from the Thermal Decomposition reactor 202c. In embodiments, the extracted gases 210c and the Carbon Dioxide (CO₂) 211c are fed through a pressure swing adsorption system to separate and purify the extracted Hydrogen (H₂), Carbon Monoxide (CO), and Carbon Dioxide (CO₂) prior to entering the Methanol production system 204c. In embodiments, separated and purified Hydrogen (H₂), Carbon Monoxide (CO), and Carbon Dioxide (CO₂) may be fed into the Methanol production system 204c individually at a rate that may optimize the production of Methanol (CH₃OH). The produced Methanol (CH₃OH) may be stored or sold. In embodiments, the Methanol production system 204c is configured to receive power from a power plant, such as the power plant system 102.

FIG. 2D schematically illustrates a representative schematic diagram of a Thermal Decomposition system 200d (“system 200d”) during steady state operations. In embodiments, the system 200d may include a Thermal Decomposition reactor 201d, a Thermal Decomposition reactor 202d, a Thermal Decomposition reactor 203d, and a Methanol production system 204d. The Thermal Decomposition reactors 201c, 202d, and 203d may each be configured to receive thermal energy to maintain the desired internal temperature, for example, from a power plant such as power plant system 102. The thermal energy may include indirect heat transfer from steam supplied by the power plant. For example, the Thermal Decomposition reactors 201c, 202d, and 203d may each include a jacket that receives the steam and transfers heat through the walls of the jacket such that no steam comes directly into contact with the interior of the reactor. In embodiments, additional thermal energy may be added to steam supplied by the power plant to indirectly heat the Thermal Decomposition reactors 201c, 202d, and 203d to the desired temperature, for example, through compression and/or heating of the steam in auxiliary compressors and/or heaters. In embodiments, auxiliary compressors and/or heaters may be powered by electrical energy from the power plant. In embodiments, the thermal energy may include heat provided to Thermal Decomposition reactors 201c, 202d, and 203d by electrical heaters powered by electrical energy from the power plant. In embodiments, the Thermal Decomposition reactors 201d, 202d, and 203d may receive any combination of steam and/or electrical heating from the power plant to maintain the desired internal temperature.

The Thermal Decomposition reactor 201d may be configured to receive Sodium Formate (HCOONa) 206 and to produce Sodium Oxalate ((COO)₂Na₂) 207 and Hydrogen (H₂) 210d. The Sodium Formate (HCOONa) 206 may be directed, via the rotating spiral 212d (e.g., first rotating spiral), into an upper portion of the Thermal Decomposition reactor 201d. The Thermal Decomposition reactor 201d may have an internal temperature of <360° C. Within the Thermal Decomposition reactor 201d, the Sodium Formate (HCOONa) 206 may undergo the reaction in Equation 4 to produce Sodium Oxalate ((COO)₂Na₂) 207 and Hydrogen (H₂) 210d:

2HCOONa→(COO)₂Na₂+H₂ (4)

In embodiments, Sodium Formate (HCOONa) 206 may be in a solid state and ground into fine powder via the rotating spiral 212d such that the Sodium Formate (HCOONa) 206 is disintegrated within the chamber. In various cases, the rotating spiral 212d may be utilized to convert the Sodium Formate (HCOONa) 206 between particles of different sizes. For example, the rotating spiral 212d may be utilized to convert the Sodium Formate (HCOONa) 206 from relatively coarser (e.g., larger) particles to relatively finer (e.g., smaller) particles. The rotating spiral 212d may be utilized to assist in the conversion of the Sodium Formate (HCOONa) 206 to the Sodium Oxalate ((COO)₂Na₂) 207. The rotating spiral 212d may be utilized to maintain the temperature in the Thermal Decomposition reactor 201d by providing a means to feed the Sodium Formate (HCOONa) 206 into the upper portion of the Thermal Decomposition reactor 201d while minimizing the potential for heat loss.

In embodiments, the Sodium Formate (HCOONa) 206, may receive thermal energy as a result of the temperature inside the Thermal Decomposition reactor 201d. The internal temperature of the Thermal Decomposition reactor 201d may cause the Sodium Formate (HCOONa) 206 to rapidly decompose into Hydrogen (H₂) 210d and the Sodium Oxalate ((COO)₂Na₂) 207. In the embodiment, the resulting Sodium Oxalate ((COO)₂Na₂) 207 sinks to the bottom of the Thermal Decomposition reactor 201d while still being thermally hot. The rotating spiral 213d (e.g., second rotating spiral) may transfer the thermally hot Sodium Oxalate ((COO)₂Na₂) 207 from the bottom of the Thermal Decomposition reactor 201d to outside the Thermal Decomposition reactor 201d for collection and/or additional industrial use and/or processing. For example, the Sodium Oxalate ((COO)₂Na₂) 207 from the bottom of the Thermal Decomposition reactor 201d may be directed, via the rotating spiral 214d (e.g., third rotating spiral), into an upper portion of the Thermal Decomposition reactor 202d. In embodiments, the rotating spiral 213d and the rotating spiral 214d may be connected via a thermally insulated conduit. The rotating spiral 213d and the rotating spiral 214d may be utilized to maintain the temperature in the Thermal Decomposition reactor 201d and the Thermal Decomposition reactor 202d by providing a means to feed the Sodium Oxalate (COO)₂Na₂) 207 into the upper portion of the Thermal Decomposition reactor 202d while minimizing the potential for heat loss. In embodiments, the rotating spiral 213d and the rotating spiral 214d may be the same rotating spiral.

The Thermal Decomposition reactor 202d may be configured to receive Sodium Oxalate ((COO)₂Na₂) 207 and to produce Sodium Carbonate (Na₂CO₃) 208 and Carbon Monoxide (CO) 211d. The Thermal Decomposition reactor 202d may have an internal temperature of about 400-450° C., for example about 440° C. Within the Thermal Decomposition reactor 202d, the Sodium Oxalate (COO)₂Na₂) 207 may undergo the reaction in Equation 5 at a temperature of about 440° C. to produce Sodium Carbonate (Na₂CO₃) 208 and the Carbon Monoxide (CO) 211d:

(COO)₂Na₂→Na₂CO₃+CO (5)

In embodiments, Sodium Oxalate ((COO)₂Na₂) 207 may be in a solid state and ground into fine powder via the rotating spiral 214d such that the Sodium Oxalate (COO)₂Na₂) 207 is disintegrated within the chamber. In various cases, the rotating spiral 214d may be utilized to convert the Sodium Oxalate ((COO)₂Na₂) 207 between particles of different sizes. For example, the rotating spiral 214d may be utilized to convert the Sodium Oxalate ((COO)₂Na₂) 207 from relatively coarser (e.g., larger) particles to relatively finer (e.g., smaller) particles. The rotating spiral 214d may be utilized to assist in the conversion of the Sodium Oxalate ((COO)₂Na₂) 207 to the Sodium Carbonate (Na₂CO₃) 208. The rotating spiral 214d may be utilized to maintain the temperature in the Thermal Decomposition reactor 202d by providing a means to feed the Oxalate ((COO)₂Na₂) 207 into the upper portion of the Thermal Decomposition reactor 202d while minimizing the potential for heat loss.

In embodiments, the Sodium Oxalate ((COO)₂Na₂) 207, may receive thermal energy as a result of the temperature inside the Thermal Decomposition reactor 202d. The internal temperature of the Thermal Decomposition reactor 202d may cause the Sodium Oxalate ((COO)₂Na₂) 207 to rapidly decompose into Carbon Monoxide (CO) 211d and the Sodium Carbonate (Na₂CO₃) 208. In the embodiment, the resulting Sodium Carbonate (Na₂CO₃) 208 sinks to the bottom of the Thermal Decomposition reactor 202d while still being thermally hot. The rotating spiral 215d (e.g., fourth rotating spiral) may transfer the thermally hot Sodium Carbonate (Na₂CO₃) 208 from the bottom of the Thermal Decomposition reactor 202d to outside the Thermal Decomposition reactor 202d for collection and/or additional industrial use and/or processing. For example, the Sodium Carbonate (Na₂CO₃) 208 from the bottom of the Thermal Decomposition reactor 202d may be directed, via the rotating spiral 216d (e.g., fifth rotating spiral), into an upper portion of the Thermal Decomposition reactor 203d. In embodiments, the rotating spiral 215d and the rotating spiral 216d may be connected via a thermally insulated conduit. The rotating spiral 215d and the rotating spiral 216d may be utilized to maintain the temperature in the Thermal Decomposition reactor 202d and the Thermal Decomposition reactor 203d by providing a means to feed the Sodium Carbonate (Na₂CO₃) 208 into the upper portion of the Thermal Decomposition reactor 203d while minimizing the potential for heat loss. In embodiments, the rotating spiral 215d and the rotating spiral 216d may be the same rotating spiral.

The Thermal Decomposition reactor 203d may be configured to receive Sodium Carbonate (Na₂CO₃) 208 and to produce Sodium Oxide (Na₂O) 209 and Carbon Dioxide (CO₂) 218d. The Thermal Decomposition reactor 203d may have an internal temperature of >800° C. Within the Thermal Decomposition reactor 203d, the Sodium Carbonate (Na₂CO₃) 208 may undergo the reaction in Equation 6 to produce Sodium Oxide (Na₂O) 209 and the Carbon Dioxide (CO₂) 218d:

Na₂CO₃→Na₂O+CO₂ (6)

In embodiments, Sodium Carbonate (Na₂CO₃) 208 may be in a solid state and ground into fine powder via the rotating spiral 216d such that the Sodium Carbonate (Na₂CO₃) 208 is disintegrated within the chamber. In various cases, the rotating spiral 216d may be utilized to convert the Sodium Carbonate (Na₂CO₃) 208 between particles of different sizes. For example, the rotating spiral 216d may be utilized to convert the Sodium Carbonate (Na₂CO₃) 208 from relatively coarser (e.g., larger) particles to relatively finer (e.g., smaller) particles. The rotating spiral 216d may be utilized to assist in the conversion of the Sodium Carbonate (Na₂CO₃) 208 to the Sodium Oxide (Na₂O) 209.

In embodiments, the Sodium Carbonate (Na₂CO₃) 208 may receive thermal energy as a result of the temperature inside the Thermal Decomposition reactor 203d. The internal temperature of the Thermal Decomposition reactor 203d may cause the Sodium Carbonate (Na₂CO₃) 208 to rapidly decompose into the Carbon Dioxide (CO₂) 218d and the Sodium Oxide (Na₂O) 209. In the embodiment, the resulting Sodium Oxide (Na₂O) 209 sinks to the bottom of the Thermal Decomposition reactor 203d while still being thermally hot. The rotating spiral 217d (e.g., sixth rotating spiral) may transfer the thermally hot Sodium Oxide (Na₂O) 209 from the bottom of the Thermal Decomposition reactor 203d to outside the Thermal Decomposition reactor 203d for collection and/or additional industrial use and/or processing. The rotating spirals 212d, 213d, 214d, 215d, 216d, and 217d may each be a metal rotating spiral (e.g., an auger), which may be located partially and/or fully in the Thermal Decomposition reactors. In embodiments, the rotating spirals 212d, 213d, 214d, 215d, 216d, and 217d may be operated by a control system utilized to control any portion of the system 200d to rotate (e.g., spin) at one or more predetermined, and/or dynamically determined (e.g., in real-time), speeds during any operation of the system 200d.

In embodiments, the Hydrogen (H₂) 210d, the Carbon Monoxide (CO) 211d, and the Carbon Dioxide (CO₂) 218d may be removed from the Thermal Decomposition reactors 201d, 202d, and 203d for collection and/or additional industrial use and/or processing. For example, a pressure swing adsorption system may be used to separate and purify the Hydrogen (H₂), the Carbon Monoxide (CO), and the Carbon Dioxide (CO₂) extracted from the Thermal Decomposition reactors for future use and/or processing. In embodiments, a portion of one or more of the Hydrogen (H₂), Carbon Monoxide (CO), and Carbon Dioxide (CO₂) extracted from the Thermal Decomposition reactors may be directed for collection and/or additional industrial use and/or processing. For example, the Hydrogen (H₂) may be directed into a portion of an electrochemical device (e.g., a hydrogen fuel cell anode) and used for producing electricity.

In embodiments, the Hydrogen (H₂) 210d, the Carbon Monoxide (CO) 211d, and the Carbon Dioxide (CO₂) 218d may be fed to the Methanol production system 204d. In embodiments, the Methanol (CH₃OH) synthesis reactions in Equations 1-3 take place within the Methanol production system 204d over a Cu/ZnO catalyst at a temperature between 200-300° C. and at a pressure between 50-100 bar. In embodiments, Hydrogen (H₂) is fed into the Methanol production system 204d from the Thermal Decomposition reactor 201d, Carbon Monoxide (CO) is fed into the Methanol production system 204d from the Thermal Decomposition reactor 202d, and Carbon Dioxide (CO₂) is fed into the Methanol production system 204d from the Thermal Decomposition reactor 203d. In embodiments, the Hydrogen (H₂) 210d, the Carbon Monoxide (CO) 211d, and the Carbon Dioxide (CO₂) 218d are fed through a pressure swing adsorption system to separate and purify the extracted Hydrogen (H₂), Carbon Monoxide (CO), and Carbon Dioxide (CO₂) prior to entering the Methanol production system 204d. In embodiments, separated and purified Hydrogen (H₂), Carbon Monoxide (CO), and Carbon Dioxide (CO₂) may be fed into the Methanol production system 204d individually at a rate that may optimize the production of Methanol (CH₃OH). The produced Methanol (CH₃OH) may be stored or sold. In embodiments, the Methanol production system 204d is configured to receive power from a power plant, such as the power plant system 102.

FIG. 3 schematically illustrates a representation of an integrated energy system (IES) 300 that includes a SMR system integrated with a Hydrogen (H₂) production system and an electrochemical device (e.g., a Hydrogen Fuel Cell). The IES 300 may include a power plant system 302, a power grid 304, a desalination system 306, a brine processing system 308, a direct air capture (DAC) system 310, a Sodium Formate (HCOONa) production system 312, a Hydrogen (H₂) production system 314, a Hydrogen (H₂) storage 316, and a Hydrogen Fuel Cell 318.

In the illustrated embodiment, the power plant system 302 may be configured to provide electrical power directly to the power grid 304. In embodiments, the power plant system 302 may produce and deliver electrical power to the power grid 304 during peak times or anytime that there is a demand for energy production. For example, when consumer energy demand imposes a high energy demand on the power grid 304 (“peak times”), the power plant system 302 may be configured to produce and provide the energy necessary for the power grid 304 to meet the high consumer demand during peak times. In embodiments, the power plant system 302 may be configured to provide energy directly to the power grid 304 as required to meet energy demand due to factors other than increased energy demand during peak times (e.g., an energy producing plant that provides energy to the power grid 304 may be offline and unable to provide energy, which creates an increased energy production demand without an increased demand for consumer electrical power).

In the illustrated embodiment, the power plant system 302 may be configured to provide steam and power to the desalination system 306. In embodiments, the desalination system 306 may be configured to utilize the steam and power from the power plant system 302 to convert supply water into a concentrated NaCl solution (“brine”), clean water, and Carbon Dioxide (CO₂). In embodiments, the brine and the clean water may be directed into the brine processing system 308 and the Carbon Dioxide (CO₂) may be directed to the DAC system 310. The brine processing system 308 may be configured to convert brine into Sodium Hydroxide (NaOH), Hydrogen (H₂), and Chlorine (Cl₂). The Sodium Hydroxide (NaOH) may be directed from the brine processing system 308 to the DAC system 310. The DAC system 310 may be configured to convert the Sodium Hydroxide (NaOH) from the brine processing system 108, the Carbon Dioxide (CO₂) from the desalination system 306, and air into Sodium Carbonate (Na₂CO₃) and Sodium Bicarbonate (NaHCO₃). In embodiments, the Sodium Carbonate (Na₂CO₃) and Sodium Bicarbonate (NaHCO₃) from the DAC system 310 may be directed into the Sodium Formate (HCOONa) production system 312. Sodium Formate (HCOONa) may be produced in a large-scale inexpensively from Formic Acid (HCOOH) via carbonylation of Methanol (CH₃OH) followed by adding water to the resulting Methyl Formate (HCOOCH₃). Sodium Formate (HCOONa) may also be produced by neutralizing Formic Acid (HCOOH) with Sodium Hydroxide (NaOH).

In embodiments, the Sodium Formate (HCOONa) production system 312 may utilize the Sodium Carbonate (Na₂CO₃) and Sodium Bicarbonate (NaHCO₃) to generate Sodium Formate (HCOONa). The Sodium Formate (HCOONa) from the Sodium Formate (HCOONa) production system 312 may be directed into the Hydrogen (H₂) production system 314. In embodiments, the Hydrogen (H₂) production system 314 may be configured to receive clean water from the desalination system 306 and Sodium Formate (HCOONa) from the Sodium Formate (HCOONa) production system 312 and produce Hydrogen (H₂). In embodiments, the Hydrogen (H₂) may be directed from the Hydrogen (H₂) storage 316 to the Hydrogen Fuel Cell 318. The Hydrogen Fuel Cell 318 may be configured to utilize the Hydrogen (H₂) from the Hydrogen (H₂) storage 316 to produce electrical power. In embodiments, the electrical power produced by the Hydrogen Fuel Cell 318 may be directed to the power grid 304.

It is understood that the IES 300 may be configured such that the power plant system 302 may simultaneously produce electrical power directly to the power grid 304 and produce Hydrogen (H₂) via the desalination system 306, the brine processing system 308, the direct air capture (DAC) system 310, the Sodium Formate (HCOONa) production system 312, and the Hydrogen (H₂) production system 314. It is also understood that the IES 300 may be configured such that the power plant system 302 may simultaneously produce and provide electrical power directly to the power grid 304 while the Hydrogen Fuel Cell 318 is utilizing Hydrogen (H₂) from the Hydrogen (H₂) storage 316 to produce and provide electrical power directly to the power grid 304.

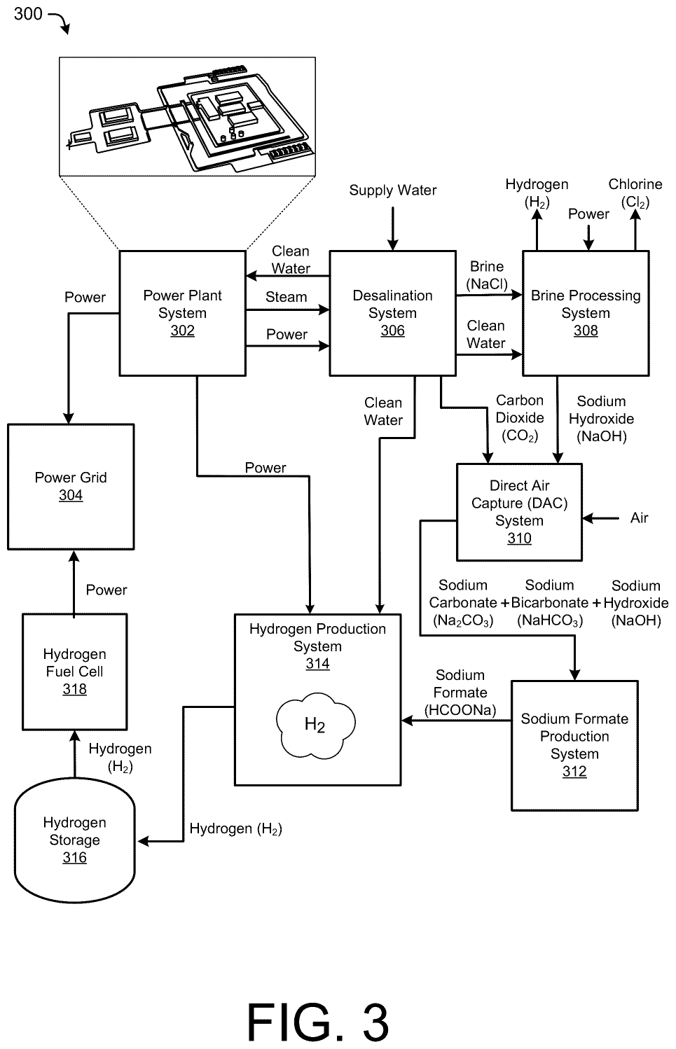

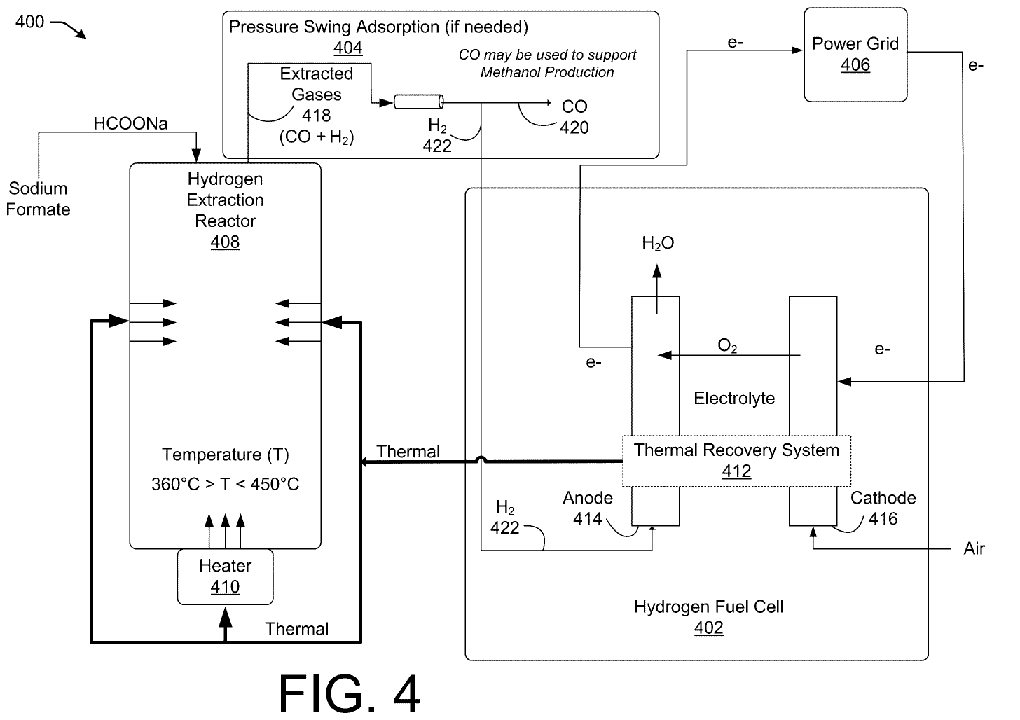

FIG. 4 illustrates a steady-state Hydrogen (H₂) production process 400 (“process 400”) utilizing Sodium Formate (HCOONa) and an electrochemical device (e.g., Hydrogen Fuel Cell 402). In embodiments, process 400 may include the Hydrogen Fuel Cell 402, the pressure swing adsorption process 404, the power grid 406, and the Hydrogen (H₂) extraction reactor 408. In embodiments, the Hydrogen (H₂) extraction reactor 408 may include the Hydrogen (H₂) extraction reactor heater 410. In embodiments, the Hydrogen Fuel Cell 402 may include the thermal recovery system 412, the anode 414, and the cathode 416.

In embodiments, Sodium Formate (HCOONa) is fed into the Hydrogen (H₂) extraction reactor 408. The Hydrogen (H₂) extraction reactor heater and the Hydrogen Fuel Cell 402 may receive heat from the thermal recovery system 412 that is powered by the power grid 406 to keep the process 400 at operational temperatures. Excess Hydrogen (H₂) that is produced may be fed back into the anode 414 of the thermal recovery system 412. Hydrogen (H₂) may also be injected from an external source (e.g., a tanker truck, a storage tank, etc.) into the Hydrogen Fuel Cell 402 to generate electricity and reduce energy from the power grid 406. The thermal energy recovered by the thermal recovery system 412 within the Hydrogen Fuel Cell 402 may be used to maintain temperature in the Hydrogen (H₂) extraction reactor 408 and disconnect grid power (i.e., the thermal energy recovered from the thermal recovery system 412 may be sufficient to maintain the Hydrogen (H₂) extraction reactor 408 at operating temperatures, thus external power may not be required to energize the Hydrogen (H₂) extraction reactor heater 410). Sodium Formate (HCOONa) may be unloaded into the Hydrogen (H₂) extraction reactor 408 for steady-state Hydrogen (H₂) generation operations.

In embodiments, the Hydrogen (H₂) extraction reactor 408 may receive Sodium Formate (HCOONa). It is understood that the Hydrogen (H₂) extraction reactor 408 may receive Sodium Formate (HCOONa) in a solid-state form or as a powder. In the illustrated embodiment, the Hydrogen (H₂) extraction reactor 408 may be maintained with an internal temperature of >360° C. and <450° C. In some embodiments, the Hydrogen (H₂) extraction reactor heater 410 may utilize electricity from the power grid 406 to maintain the Hydrogen (H₂) extraction reactor internal temperature. In some embodiments, the Hydrogen (H₂) extraction reactor heater 410 may utilize thermal energy recovered from the thermal recovery system 412 to maintain the Hydrogen (H₂) extraction reactor internal temperature.

During its operation, the Hydrogen (H₂) extraction reactor 408 may process Sodium Formate (HCOONa) to produce extracted gases 418 (e.g., Carbon Monoxide (CO) and Hydrogen (H₂). It is understood that the extracted gases 418 may be a mixture of gases with varied concentrations. The extracted gases 418 may be directed to the pressure swing adsorption process 404 to be separated into separate gases (i.e., a mixture of Hydrogen (H₂) and Carbon Monoxide (CO) may be separated by the pressure swing adsorption process 404).

In embodiments, the Carbon Monoxide (CO) 420 may be used for Methanol (CH₃OH) production. In embodiments, the Hydrogen (H₂) 422 may be directed to the Hydrogen Full Cell 402. It is understood the process 400 may use a Hydrogen (H₂) fuel cell other than the type depicted within FIG. 4. In the illustrated embodiment, the Hydrogen (H₂) 422 may be directed to the anode 414 of the Hydrogen Fuel Cell 402 to be oxidized and separated into negatively charged hydrogen electrons and positively charged hydrogen ions. The negatively charged hydrogen electrons may be directed to the power grid 406 to generate electricity before being directed to the cathode 416. In embodiments, air (e.g., atmospheric air containing Oxygen (O₂) may be directed into the cathode 416 of the Hydrogen Fuel Cell 402. The Oxygen (O₂) in the cathode 416 may combine with the negatively charged electrons directed into in the cathode 416 from the power grid 406 and the positively charged hydrogen particles that traveled from the anode 414 to the cathode 416 via the electrolyte to generate water (H₂O). The oxidation of the Hydrogen (H₂) 422 and generation of water (H₂O) in the Hydrogen Fuel Cell 402 may produce heat. In embodiments, the thermal recovery system 412 may capture the heat generated by the Hydrogen Fuel Cell 402 and direct the heat to Hydrogen (H₂) extraction reactor 408.

It is understood that the thermal recovery system 412 may utilize a thermal fluid to transfer heat, a Stirling engine with an electrical component to power a heater, or other system components suitable to recover and reuse the heat generated by the Hydrogen Fuel Cell 402.

For example, the Hydrogen (H₂) extraction reactor heater and the hydrogen fuel cell may receive heat from the thermal recovery system that is powered by the power grid to keep the system at operational temperatures. Excess Hydrogen (H₂) that is produced may be fed back into the anode of the thermal recovery system. The recovered thermal from the hydrogen fuel cell may be used to maintain temperature in the Hydrogen (H₂) extraction reactor and disconnect grid power. Pressure swing absorption may also be used. Carbon Monoxide (CO) may be used to support Methanol (CH₃OH) production.

In embodiments, the process 400 may include the Hydrogen Fuel Cell 402, the Hydrogen (H₂) extraction reactor 408, and the power grid 406. In embodiment, the Hydrogen Fuel Cell 402 may include the anode 414, the cathode 416, the heater 410, and the thermal recovery system 412. In embodiments, the Hydrogen (H₂) extraction reactor 408 may include the Hydrogen (H₂) extraction reactor heater 410. In embodiments, the anode 414 may be configured to receive Hydrogen (H₂) from the Hydrogen (H₂) supply 422. It is understood that the Hydrogen (H₂) supply 422 may include a Hydrogen (H₂) production system, a permanent Hydrogen (H₂) storage tank, and/or a temporary Hydrogen (H₂) storage tank (e.g., a Hydrogen (H₂) fuel truck, portable tank(s), etc.). In an embodiment, the Hydrogen Fuel Cell 402 may only need approximately 2 kg of Hydrogen (H₂) from an external source during startup. It is understood that in embodiments, external Hydrogen (H₂) may only be required for the Hydrogen Fuel Cell 402 during startup, and that after startup, the Hydrogen Fuel Cell 402 may receive necessary Hydrogen (H₂) from the Hydrogen (H₂) extraction reactor 408.

In embodiments, the power grid 406 may provide electrical power to the heater 410 and the Hydrogen (H₂) extraction reactor heater 410. It is understood that the heater 410 and the Hydrogen (H₂) extraction reactor heater 410 may only require the use of electrical power from the power grid 406 during startup. In some embodiments, electrical power may only be required for the heater 410 during startup, and that after startup, the Hydrogen Fuel Cell 402 may no longer require the use of the heater 410. For example, in embodiments, the Hydrogen Fuel Cell 402 may use the heater 410 during startup because the normal steady-state operation of the Hydrogen Fuel Cell 402 may generate the heat necessary to sustain the operation of the Hydrogen Fuel Cell 402.

In some embodiments, electrical power may only be required for the Hydrogen (H₂) extraction reactor heater 410 during startup, and that after startup, the Hydrogen (H₂) extraction reactor 408 may no longer require the use of the Hydrogen (H₂) extraction reactor heater 410. For example, in embodiments, the Hydrogen (H₂) extraction reactor heater 410 may use electrical energy to provide the heat necessary for the operation of the Hydrogen (H₂) extraction reactor 408 during startup. In embodiment, the normal steady-state operation of the Hydrogen Fuel Cell 402 may generate heat, which may be recovered by the thermal recovery system 412 and transferred to the Hydrogen (H₂) extraction reactor 408. During normal steady-state operation, the heat recovered by the thermal recovery system 412 may be adequate for operation of the Hydrogen (H₂) extraction reactor 408.

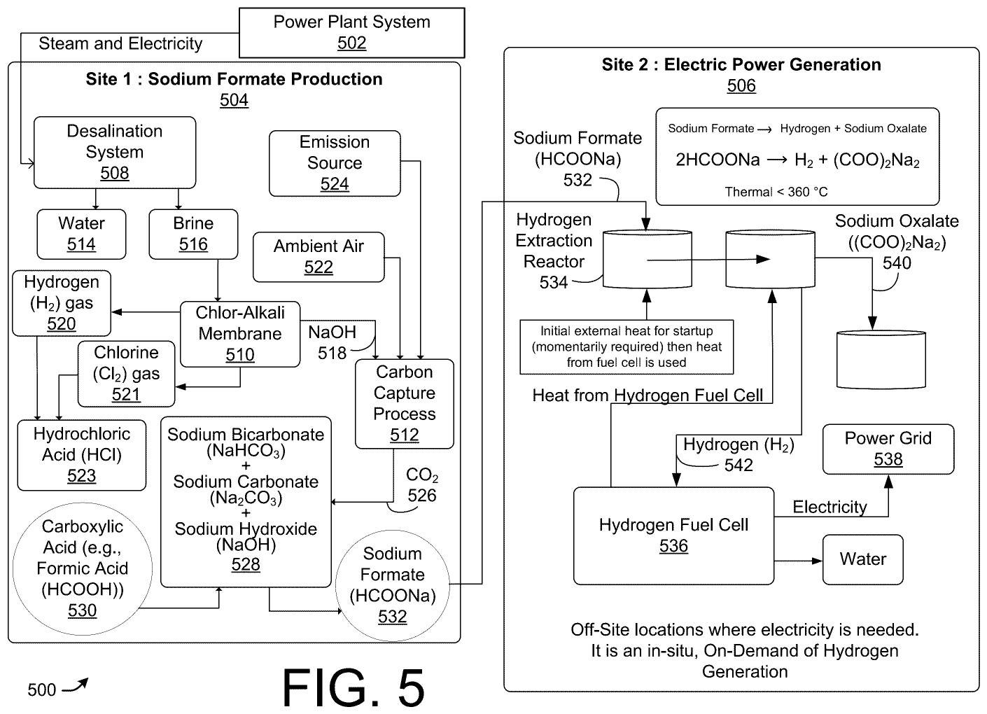

FIG. 5 illustrates in-situ and on-demand Hydrogen (H₂) production system (“system 500”) to support emergency and limited energy imbalance market (EIM) using Sodium Formate (HCOONa). In embodiments, the system 500 may include a power plant system 502, a first site 504, and a second site 506. The first site 504 may be used for Sodium Formate (HCOONa) production. The second site 506 may be an off-site location (i.e., located wherever electricity is needed that may not be in proximity to the power plant system 500). The second site 506 may provide in-situ on-demand Hydrogen (H₂) generation. In embodiments, the power plant system 500 may include the SMR system.

In embodiments, the first site 504 may be used for Sodium Formate (HCOONa) production. The first site 504 may include the desalination system 508, the chlor-alkali membrane 510, and the carbon capture process 512. It is understood the chlor-alkali membrane 510 may include any type of electrolysis system and/or process configured to process brine into Sodium Hydroxide (NaOH). At the first site 504, the power plant system 502 may supply steam and electricity to the desalination system 508. The desalination system 508 may produce water 514 and brine 516 (i.e., a concentrated Sodium Chloride (NaCl) solution). The brine 516 may be directed into the chlor-alkali membrane 510. The chlor-alkali membrane 510 may be configured to receive the brine 516 and generate Sodium Hydroxide (NaOH) 518, Hydrogen (H₂) gas 520, and Chlorine (Cl₂) gas 521 via electrolysis. In embodiments, the Hydrogen (H₂) gas 520 and the Chlorine (Cl₂) gas 521 may be combined to form Hydrochloric Acid (HCl) 523. In embodiments, the production of Hydrochloric Acid (HCl) may be represented by Equation 7 below:

H₂+Cl₂→2HCL (7)

In embodiments, the carbon capture process 512 may receive ambient air 522 (e.g., atmospheric air containing Carbon Dioxide (CO₂)) and/or an emission source 524 (e.g., gases containing Carbon Dioxide (CO₂) released as an emission from a process, machine, device, etc.) and produce Carbon Dioxide (CO₂) 526, which may be useful for industrial processes. In embodiments, the Sodium Hydroxide (NaOH) 518 may be combined with the Carbon Dioxide (CO₂) 526 to generate Sodium Bicarbonate (NaHCO₃) and Sodium Carbonate (Na₂CO₃) 528. In embodiments, a carboxylic acid (e.g., Formic Acid (HCOOH)) 530 may be reacted with the Sodium Bicarbonate (NaHCO₃) and Sodium Carbonate (Na₂CO₃) 528 to produce Sodium Formate (HCOONa) 532 that may be transported to the second site 506.

In embodiments, the second site 506 may include the Hydrogen (H₂) extraction reactor 534, an electrochemical device (e.g., Hydrogen Fuel Cell 536), and the power grid 538. In embodiments, the Hydrogen (H₂) extraction reactor 534 may receive Sodium Formate (HCOONa) 532 to produce Sodium Oxalate ((COO)₂Na₂) 540 and Hydrogen (H₂) 542. In embodiments, the conversion of the Sodium Formate (HCOONa) 532 to Sodium Oxalate ((COO)₂Na₂) 540 and Hydrogen (H₂) 542 may be represented by the reaction in Equation 4:

2HCOONa→(COO)₂Na₂+H₂ (4)

The Hydrogen (H₂) 542 may be directed to the Hydrogen Fuel Cell 536, which may be configured to convert the Hydrogen (H₂) 542 into electricity and water. It is understood that the Hydrogen (H₂) 542 may be directly directed to the Hydrogen Fuel Cell 536 and/or directed to a Hydrogen (H₂) tank (i.e., tanker truck, portable storage tank, permanently installed tank, etc.). The electricity produced by the Hydrogen Fuel Cell 536 may be directed to the power grid 538. In some embodiments, the Hydrogen Fuel Cell 536 may operate to produce electricity as needed to support an EIM. In embodiments, the Hydrogen Fuel Cell 536 may generate heat during operation which may be directed to the Hydrogen (H₂) extraction reactor 534.

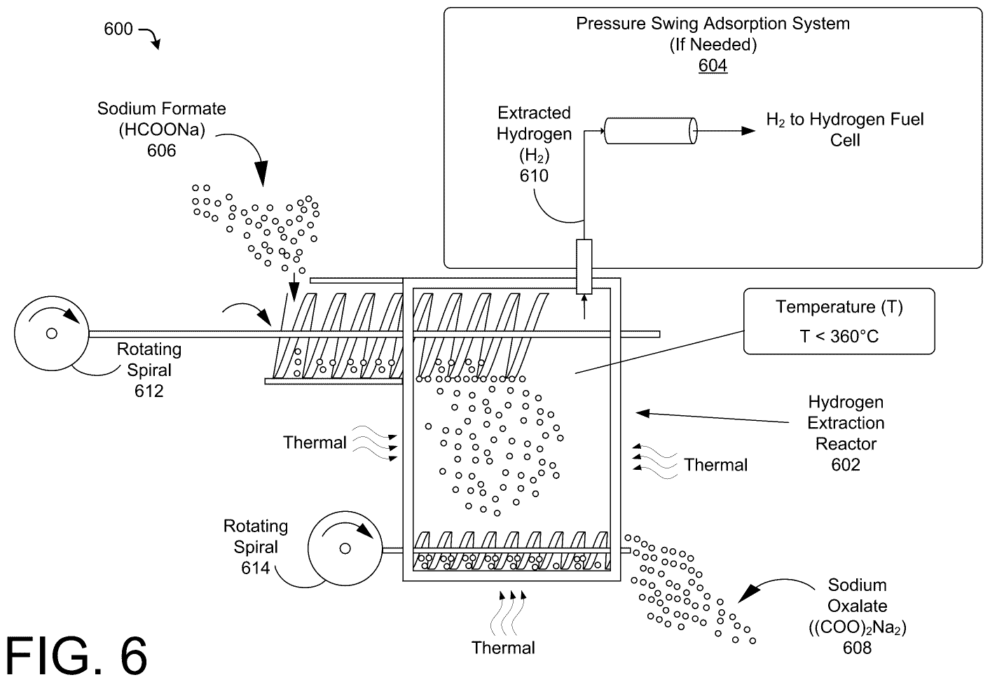

FIG. 6 schematically illustrates a representative schematic diagram of a Hydrogen (H₂) extraction reactor system 600 (“system 600”) during steady state operations. In embodiments, the system 600 may include the Hydrogen (H₂) extraction reactor 602 and a pressure swing adsorption system 604. The Hydrogen (H₂) extraction reactor 602 may be configured to receive Sodium Formate (HCOONa) 606 and produce Sodium Oxalate (COO)₂Na₂) 608 and/or extracted Hydrogen (H₂) 610. The Sodium Formate (HCOONa) 606 may be directed, via the rotating spiral 612 (e.g., first rotating spiral), into an upper portion of the Hydrogen (H₂) extraction reactor 602. The Hydrogen (H₂) extraction reactor 602 may have an internal temperature <360° C.

In various cases, the rotating spiral 612 may be utilized to convert the Sodium Formate (HCOONa) 606 between particles of different sizes. For example, the rotating spiral 612 may be utilized to convert the Sodium Formate (HCOONa) 606 from relatively coarser (e.g., larger) particles to relatively finer (e.g., smaller) particles. The rotating spiral 612 may be utilized to assist in a conversion between the Sodium Formate (HCOONa) 606 to the Sodium Oxalate ((COO)₂Na₂) 608. The rotating spiral 612 may be utilized to maintain the temperature in the Hydrogen (H₂) extraction reactor 602 by providing a means to feed the Sodium Formate (HCOONa) 606 into the upper portion of the Hydrogen (H₂) extraction reactor 602 while minimizing the potential for heat loss. The rotating spiral 612 may be a metal rotating spiral (e.g., an auger), which may be located partially and/or fully in the first Hydrogen (H₂) extraction reactor 602. In embodiments, the rotating spiral 612 may be operated by a control system utilized to control any portion of the system 600 to rotate (e.g., spin) at one or more predetermined, and/or dynamically determined (e.g., in real-time), speeds during any operation of the system 600.

In an embodiment, the Sodium Formate (HCOONa) 606, may receive thermal energy as a result of the temperature inside the Hydrogen (H₂) extraction reactor 602. The internal temperature of the Hydrogen (H₂) extraction reactor 602 may cause the Sodium Formate (HCOONa) 606 to rapidly decompose into the extracted Hydrogen (H₂) 610 and the Sodium Oxalate ((COO)₂Na₂) 608. In the embodiment, the extracted Hydrogen (H₂) 610 may be produced instantaneously following the decomposition of the Sodium Formate (HCOONa) 606 into the Sodium Oxalate ((COO)₂Na₂) 608. In the embodiment, the resulting Sodium Oxalate ((COO)₂Na₂) 608 sinks to the bottom of the Hydrogen (H₂) extraction reactor 602 while still being thermally hot. The rotating spiral 614 (e.g., second rotating spiral) may transfer the thermally hot Sodium Oxalate ((COO)₂Na₂) 608 from the bottom of the Hydrogen (H₂) extraction reactor 602 to outside the Hydrogen (H₂) extraction reactor 602 for collection and/or additional industrial processing.

In embodiments, the pressure swing adsorption system 604 may be used to purify the extracted Hydrogen (H₂) 610 for future processing. In embodiments, the extracted Hydrogen (H₂) 610 may be directed into a hydrogen fuel cell and used for producing electricity.

For example, Sodium Formate (HCOONa) is fed into an extraction reactor chamber. In some embodiments, Sodium Formate (HCOONa) may be in a solid state and ground into fine powder via a rotating spiral such that the Sodium Formate (HCOONa) is disintegrated within the chamber. The recovered thermal maintains the temperature inside the extraction reactor chamber at <360° C. and Sodium Oxalate (COO)₂Na₂) is produced. The reactor chamber may be coupled to a pressure swing absorption system to purify the Hydrogen (H₂) gas to be fed into the hydrogen fuel cell.

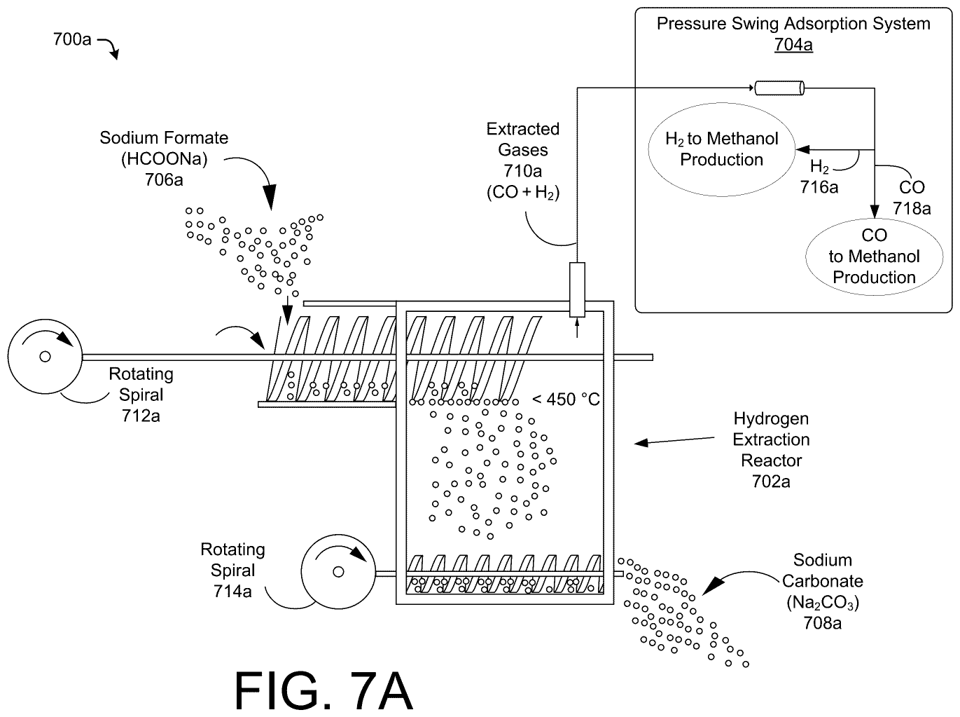

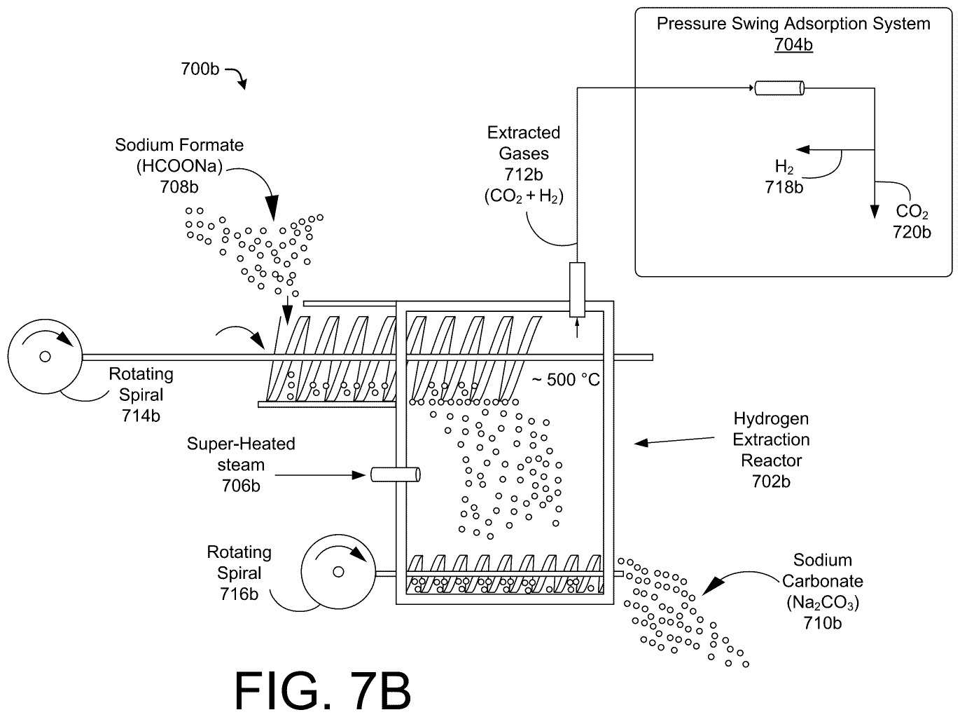

FIG. 7A schematically illustrates a representative schematic diagram of a Hydrogen (H₂) extraction reactor system 700a (“system 700a”) during steady state operations for the thermal decomposition of Sodium Formate (HCOONa) (i.e., dry process). In embodiments, the system 700a may include the Hydrogen (H₂) extraction reactor 702a and a pressure swing adsorption system 704a. The Hydrogen (H₂) extraction reactor 702a may be configured to receive Sodium Formate (HCOONa) 706a and produce Sodium Carbonate (Na₂CO₃) 708a and/or extracted gases 710a. The Sodium Formate (HCOONa) 706a may be directed, via the rotating spiral 712a (e.g., first rotating spiral), into an upper portion of the Hydrogen (H₂) extraction reactor 702a. The Hydrogen (H₂) extraction reactor 702a may have an internal temperature of <450° C.

In various cases, the rotating spiral 712a may be utilized to convert the Sodium Formate (HCOONa) 706a between particles of different sizes. For example, the rotating spiral 712a may be utilized to convert the Sodium Formate (HCOONa) 706a from relatively coarser (e.g., larger) particles to relatively finer (e.g., smaller) particles. The rotating spiral 712a may be utilized to assist in a conversion between the Sodium Formate (HCOONa) 706a to the Sodium Carbonate (Na₂CO₃) 708a. The rotating spiral 712a may be utilized to maintain the temperature in the Hydrogen (H₂) extraction reactor 702a by providing a means to feed the Sodium Formate (HCOONa) 706a into the upper portion of the Hydrogen (H₂) extraction reactor 702a while minimizing the potential for heat loss. The rotating spiral 712a may be a metal rotating spiral (e.g., an auger), which may be located partially and/or fully in the first Hydrogen (H₂) extraction reactor 702a. In embodiments, the rotating spiral 712a may be operated by a control system utilized to control any portion of the system 700a to rotate (e.g., spin) at one or more predetermined, and/or dynamically determined (e.g., in real-time), speeds during any operation of the system 700a.

In an embodiment, the Sodium Formate (HCOONa) 706a, may receive thermal energy as a result of the temperature inside the Hydrogen (H₂) extraction reactor 702a. The Hydrogen (H₂) extraction reactor 702a may be configured to receive super-heated steam and Sodium Formate (HCOONa) 708b and produce Sodium Carbonate (Na₂CO₃) and/or extracted gases. It is understood that the super-heated steam may be steam (e.g., process steam) from an SMR system. The internal temperature of the Hydrogen (H₂) extraction reactor 702a may cause the Sodium Formate (HCOONa) 706a to rapidly decompose into the extracted gases 710a and the Sodium Carbonate (Na₂CO₃) 708a. In the embodiment, the extracted gases 710a may be produced instantaneously following the decomposition of the Sodium Formate (HCOONa) 706a into the Sodium Carbonate (Na₂CO₃) 708a. In the embodiment, the resulting Sodium Carbonate (Na₂CO₃) 708a sinks to the bottom of the Hydrogen (H₂) extraction reactor 702a while still being thermally hot. The rotating spiral 714a (e.g., second rotating spiral) may transfer the thermally hot Sodium Carbonate (Na₂CO₃) 708a from the bottom of the Hydrogen (H₂) extraction reactor 702a (i.e., the lower portion of the Hydrogen (H₂) extraction reactor) to outside the Hydrogen (H₂) extraction reactor 702a for collection and/or additional industrial processing.

In embodiments, the extracted gases 710a may include a mixture of Carbon Monoxide (CO) and Hydrogen (H₂). The pressure swing adsorption system 704 may be used to separate the extracted gases 710a into Hydrogen (H₂) 716a and Carbon Monoxide (CO) 718a. In embodiments, the Hydrogen (H₂) 716a and the Carbon Monoxide (CO) 718a may be used for Methanol (CH₃OH) production.

Basic ingredients and infrastructures for Methanol (CH₃OH) production include Hydrogen (H₂) and Carbon Monoxide (CO), as demonstrated by the reaction in Equation 2:

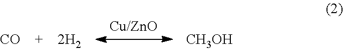

In various embodiments, Copper (Cu) and/or Zinc Oxide (ZnO) is used as a catalyst, at 200° C.-300° C. and 50-100 bar.

FIG. 7B schematically illustrates a representative schematic diagram of a Hydrogen (H₂) extraction reactor system 700b (“system 700b”) during steady state operations for the hydrothermal decomposition of Sodium Formate (HCOONa) (i.e., wet process). In embodiments, the system 700b may include the Hydrogen (H₂) extraction reactor 702b and a pressure swing adsorption system 704b. The Hydrogen (H₂) extraction reactor 702b may be configured to receive super-heated steam 706b and Sodium Formate (HCOONa) 708b and produce Sodium Carbonate (Na₂CO₃) 710b and/or extracted gases 712b. The Sodium Formate (HCOONa) 708b may be directed, via the rotating spiral 714b (e.g., first rotating spiral), into an upper portion of the Hydrogen (H₂) extraction reactor 702b. The Hydrogen (H₂) extraction reactor 702b may have an internal temperature of about 800° C. It is understood that the super-heated steam 706b may be steam (e.g., process steam) from an SMR system.

In various cases, the rotating spiral 714b may be utilized to convert the Sodium Formate (HCOONa) 708b between particles of different sizes. For example, the rotating spiral 714b may be utilized to convert the Sodium Formate (HCOONa) 708b from relatively coarser (e.g., larger) particles to relatively finer (e.g., smaller) particles. The rotating spiral 714b may be utilized to assist in a conversion between the Sodium Formate (HCOONa) 708b to the Sodium Carbonate (Na₂CO₃) 710b. The rotating spiral 714b may be utilized to maintain the temperature in the Hydrogen (H₂) extraction reactor 702b by providing a means to feed the Sodium Formate (HCOONa) 708b into the upper portion of the Hydrogen (H₂) extraction reactor 702b while minimizing the potential for heat loss. The rotating spiral 714b may be a metal rotating spiral (e.g., an auger), which may be located partially and/or fully in the first Hydrogen (H₂) extraction reactor 702b. In embodiments, the rotating spiral 714b may be operated by a control system utilized to control any portion of the system 700b to rotate (e.g., spin) at one or more predetermined, and/or dynamically determined (e.g., in real-time), speeds during any operation of the system 700b.

In an embodiment, the Sodium Formate (HCOONa) 708b, may receive thermal energy as a result of the temperature inside the Hydrogen (H₂) extraction reactor 702b. The internal temperature of the Hydrogen (H₂) extraction reactor 702b may cause the Sodium Formate (HCOONa) 708b to rapidly decompose into the extracted gases 712b and the Sodium Carbonate (Na₂CO₃) 710b. In the embodiment, the extracted gases 712b may be produced instantaneously following the decomposition of the Sodium Formate (HCOONa) 708b into the Sodium Carbonate (Na₂CO₃) 710b. In the embodiment, the resulting Sodium Carbonate (Na₂CO₃) 710b sinks to the bottom of the Hydrogen (H₂) extraction reactor 702b while still being thermally hot. The rotating spiral 716b (e.g., second rotating spiral) may transfer the thermally hot Sodium Carbonate (Na₂CO₃) 710b from the bottom of the Hydrogen (H₂) extraction reactor 702b (i.e., the lower portion of the Hydrogen (H₂) extraction reactor) to outside the Hydrogen (H₂) extraction reactor 702b for collection and/or additional industrial processing.

In embodiments, the extracted gases 712b may include a mixture of Carbon Dioxide (CO₂) and Hydrogen (H₂). The pressure swing adsorption system 704b may be used to separate the extracted gases 712b into Hydrogen (H₂) 718a and Carbon Dioxide (CO₂) 720b. In embodiments, the Hydrogen (H₂) 718b and the Carbon Dioxide (CO₂) 720b may be used for future processing.

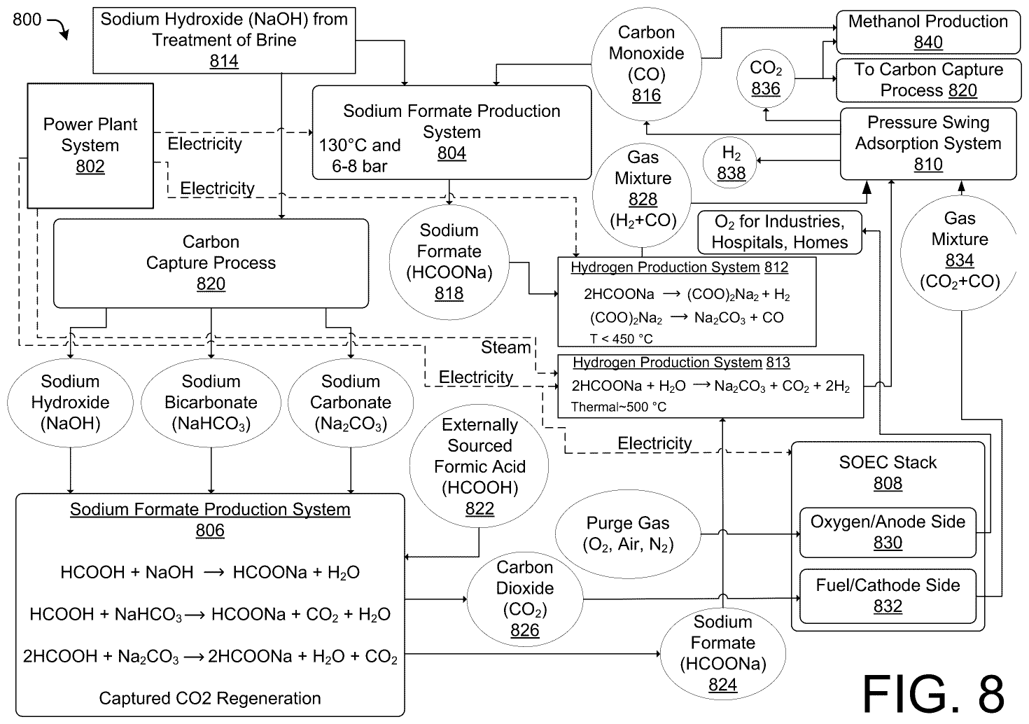

FIG. 8 illustrates Hydrogen (H₂) production process 800 (“process 800”) that uses multiple Sodium Formate (HCOONa) production systems simultaneously. The process 800 may include the power plant system 802, the Sodium Formate (HCOONa) production system 804 (e.g., first Sodium Formate (HCOONa) production system) and the Sodium Formate (HCOONa) production system 806 (e.g., second Sodium Formate (HCOONa) production system), an electrochemical device, such as a co-electrolysis system (e.g., solid oxide electrolysis cell (SOEC) stack 808), the pressure swing adsorption system 810, the Hydrogen (H₂) production system 812, and the Hydrogen (H₂) production system 813. In embodiments, the power plant system 802 may provide electricity to the Sodium Formate (HCOONa) production system 804, the Hydrogen (H₂) production system 812, the Hydrogen (H₂) production system 813, and the SOEC stack 808. In embodiments, the power plant system 802 may include an SMR system.

In embodiments, the Sodium Formate (HCOONa) production system 804 may be configured to receive Sodium Hydroxide (NaOH) 814 (e.g., first Sodium Hydroxide (NaOH)) that may be produced via treatment of brine (e.g., treatment of brine produced by a desalination system) and Carbon Monoxide (CO) 816 produced by the pressure swing adsorption system 810 to produce Sodium Formate (HCOONa) 818. In embodiments, the Sodium Formate (HCOONa) production system 804 may have operating parameters including 130° C. and a pressure range of 6-8 Bar. Production of Sodium Formate (HCOONa) 818 via the Sodium Formate (HCOONa) production system 804 may be represented by the following reaction in Equation 8:

NaOH(s)+CO→HCOONa (8)

In embodiments, the Sodium Formate (HCOONa) 818 may be directed into the Hydrogen (H₂) production system 812.

In embodiments, the Sodium Formate (HCOONa) production system 806 may be configured to receive Sodium Hydroxide (NaOH) (e.g., second Sodium Hydroxide (NaOH)), Sodium Bicarbonate (NaHCO₃), and Sodium Carbonate (Na₂CO₃) from the carbon capture process 820, and externally sourced Formic Acid (HCOOH) 822 to produce Sodium Formate (HCOONa) 824 and Carbon Dioxide (CO₂) 826. The Sodium Formate (HCOONa) 824 may be directed to the Hydrogen (H₂) production system 813. In embodiments, the production of Sodium Formate (HCOONa) 824 via the Sodium Formate (HCOONa) production system 806 using Formic Acid (HCOOH) from an external supply and a solution of Sodium Hydroxide (NaOH), Sodium Bicarbonate (NaHCO₃), and Sodium Carbonate (Na₂CO₃) from the carbon capture process 820 (e.g., DAC unit) may be represented by the following reactions in Equations 9-11:

HCOOH+NaOH→HCOONa+H₂O (9)

2HCOOH+Na₂CO₃-2HCOONa+H₂O+CO₂ (10)

HCOOH+NaHCO₃→HCOONa+H₂O+CO₂ (11)

In embodiments, the Hydrogen (H₂) production system 812 may have an operating temperature range of 360° C. and less than 450° C. to produce gas mixture 828. Production of gas mixture 828 via the Hydrogen (H₂) production system 812 may be represented by the reactions in Equations 4 and 5:

2HCOONa→(COO)₂Na₂+H₂ (4)

(COO)₂Na₂→Na₂CO₃+CO (5)

The gas mixture 828 may include Hydrogen (H₂) and Carbon Monoxide (CO), and the gas mixture 828 may be directed to the pressure swing adsorption system 810 for processing.

In embodiments, the Hydrogen (H₂) production system 813 may be configured to receive process steam from the power plant system 802. The Hydrogen (H₂) production system 813 may have an operating temperature of approximately 800° C. to produce a mixture of Carbon Dioxide (CO₂) and Hydrogen (H₂). Production of the Carbon Dioxide (CO₂) and the Hydrogen (H₂) via the Hydrogen (H₂) production system 813 may be represented by the following Equation 12:

2HCOONa+H₂O(steam)→Na₂CO₃₊₂H₂+CO₂ (12)

The Carbon Dioxide (CO₂) and the Hydrogen (H₂) produced by the Hydrogen (H₂) production system 813 may be directed to the pressure swing adsorption system 810 for processing.

In embodiments, the stack 808 may include the Oxygen/anode side 830 and the fuel/cathode side 832. The Oxygen/anode side 830 may receive purge gas (e.g., Oxygen (O₂), atmospheric air, Nitrogen (N₂), etc.). The fuel/cathode side 832 may receive the Carbon Dioxide (CO₂) 826 produced by the Sodium Formate (HCOONa) production system 806. The SOEC stack 808 may produce Oxygen (O₂) for use in hospitals, homes, and other industries. The SOEC stack 808 may produce gas mixture 834. In embodiments, the gas mixture 834 may include Carbon Monoxide (CO) and Carbon Dioxide (CO₂). The gas mixture 834 may be directed to the pressure swing adsorption system 810.

In embodiments, the pressure swing adsorption system 810 may be configured to receive the gas mixture 834 from the SOEC stack 808, the gas mixture 828 from the Hydrogen (H₂) production system 812, and Carbon Dioxide (CO₂) and Hydrogen (H₂) from the Hydrogen (H₂) Production system 813. The pressure swing adsorption system 810 may produce the Carbon Monoxide (CO) 816, the Carbon Dioxide (CO₂) 836, and the Hydrogen (H₂) 838. The Carbon Monoxide (CO) 816 may be directed to the Sodium Formate (HCOONa) production system 804 to be used to produce Sodium Formate (HCOONa) and/or to the Methanol (CH₃OH) production system 840 to combine with Hydrogen (H₂) for the production of Methanol (CH₃OH). The Carbon Dioxide (CO₂) 836 may be directed to the carbon capture process 820 to be recaptured and reused, and/or the Carbon Dioxide (CO₂) 836 may be used for Methanol (CH₃OH) production by the Methanol (CH₃OH) production system 840. The Hydrogen (H₂) 838 may be used to produce electricity (e.g., directed to a hydrogen fuel cell) to help manage an EIM, and/or collected for storage (e.g., permanent tank, portable tank, etc.).

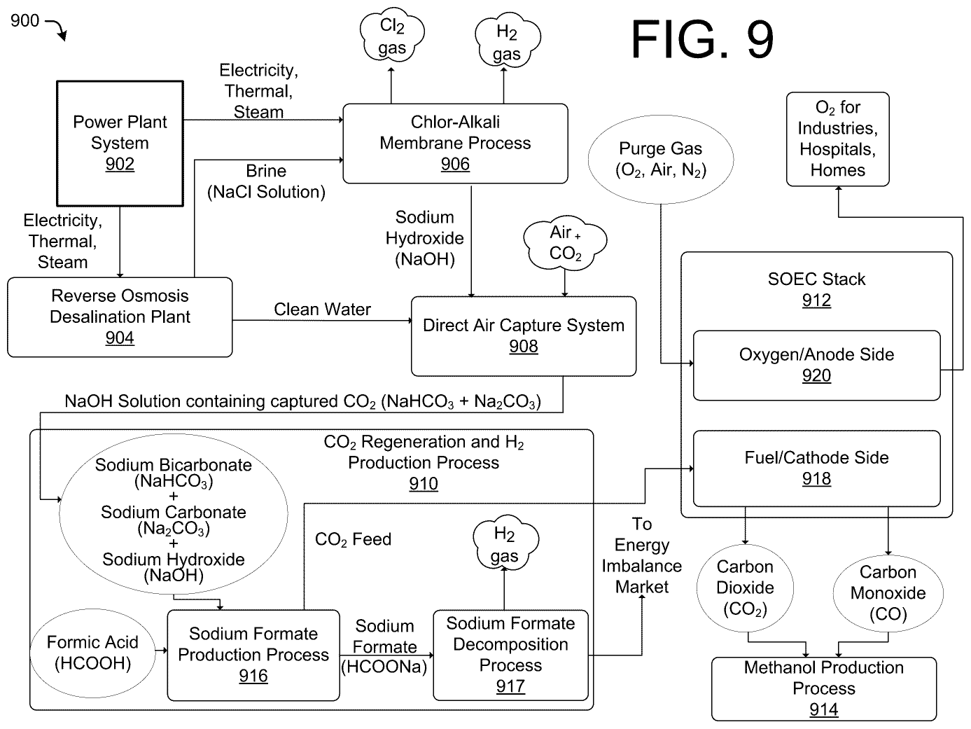

FIG. 9 illustrates an integrated energy system (IES) 900 configured to capture and produce Carbon Dioxide (CO₂) and Carbon Monoxide (CO) for use in Methanol (CH₃OH) production. In embodiments, the IES 900 may include the power plant system 902, the reverse osmosis desalination plant (“desalination plant”) 904, the chlor-alkali membrane process 906, the direct air capture (“DAC”) system 908, the CO₂ regeneration and H₂ production process 910, an electrochemical device, such as a co-electrolysis system (e.g., the SOEC stack 912), and the Methanol (CH₃OH) production process 914. In embodiments, the power plant system 902 may include a SMR system. The power plant system 902 may provide electricity, thermal, and steam to the desalination plant 904 and the chlor-alkali membrane process 906. The desalination plant 904 may provide clean water to the DAC system 908 and may provide brine (NaCl solution) to the chlor-alkali membrane process 906. The chlor-alkali membrane process 906 may produce Sodium Hydroxide (NaOH), Chlorine (Cl₂) gas, and Hydrogen (H₂) gas. The Sodium Hydroxide (NaOH) may be directed to the DAC system 908.

In embodiments, the DAC system 908 may be configured to receive clean water from the desalination plant 904, Sodium Hydroxide (NaOH) from the chlor-alkali membrane process 906, and air from the atmosphere (e.g., atmospheric Carbon Dioxide (CO₂)), and produce Sodium Carbonate (Na₂CO₃) and Sodium Bicarbonate (NaHCO₃) (e.g., an NaOH solution containing the captured Carbon Dioxide (CO₂)). The Sodium Carbonate (Na₂CO₃), the Sodium Bicarbonate (NaHCO₃), and the Sodium Hydroxide (NaOH) solution may be directed to the Carbon Dioxide (CO₂) and Hydrogen (H₂) production process 910.

In embodiments, the Sodium Carbonate (Na₂CO₃), the Sodium Bicarbonate (NaHCO₃), and the Sodium Hydroxide (NaOH) solution may be combined with externally sourced Formic Acid (HCOOH) in the Sodium Formate (HCOONa) production process 916 to produce Sodium Formate (HCOONa) and Carbon Dioxide (CO₂). The Sodium Formate (HCOONa) may be directed to the Sodium Formate Decomposition Process 917. In embodiments, the production of Sodium Formate (HCOONa) via the Sodium Formate (HCOONa) production system 916 using Formic Acid (HCOOH) from an external supply and a solution of Sodium Hydroxide (NaOH), Sodium Bicarbonate (NaHCO₃), and Sodium Carbonate (Na₂CO₃) from the DAC system 908 may be represented by the following reactions in Equations 9-11:

HCOOH+NaOH→HCOONa+H₂O (9)

2HCOOH+Na₂CO₃-2HCOONa+H₂O+CO₂ (10)

HCOOH+NaHCO₃-HCOONa+H₂O+CO₂ (11)

In embodiments, the Sodium Formate (HCOONa) from the Sodium Formate (HCOONa) production process 916 may be directed to the Sodium Formate (HCOONa) decomposition process 917. In embodiments, the Sodium Formate (HCOONa) may be decomposed either thermally or hydrothermally, which may produce Hydrogen (H₂) that may be stored or sent to an EIM to assist with energy production. In embodiments, the Carbon Dioxide (CO₂) produced in the Sodium Formate (HCOONa) production process 916 may be directed to the fuel/cathode side 918 of the SOEC stack 912. The SOEC stack 912 may include the Oxygen (O₂)/Anode side 920. In embodiments, the Oxygen (O₂)/Anode side 920 may be configured to receive purge gas (e.g., Oxygen (O₂), air, Nitrogen (N₂), etc.). The SOEC stack 912 may be configured to produce Oxygen (O₂) for a variety of uses (e.g., hospitals, homes, other industries, etc.). The SOEC stack 912 may be configured to produce Carbon Monoxide (CO) and Carbon Dioxide (CO₂) that may be directed to the Methanol (CH₃OH) production process 914.

For example, a nuclear reactor system may provide electricity, thermal, and/or steam to support reverse osmosis desalination plant where brine treatment is provided, and a chlor-alkali membrane process is conducted in a separate plant. The electricity, thermal, and steam from the nuclear reactor system may also be provided to the chlor-alkali membrane process. From the chlor-alkali membrane process, Chlorine (Cl₂) and Hydrogen (H₂) gas are produced. Resulting Sodium Hydroxide (NaOH) from the chlor-alkali membrane process and clean water from desalination can be fed into DAC system. The NaOH solution containing captured carbon dioxide is processed into Sodium Carbonate (Na₂CO₃) and Sodium Bicarbonate (NaHCO₃). Sodium Formate (HCOONa) may be then applied to a pathway for thermal decomposition described above.

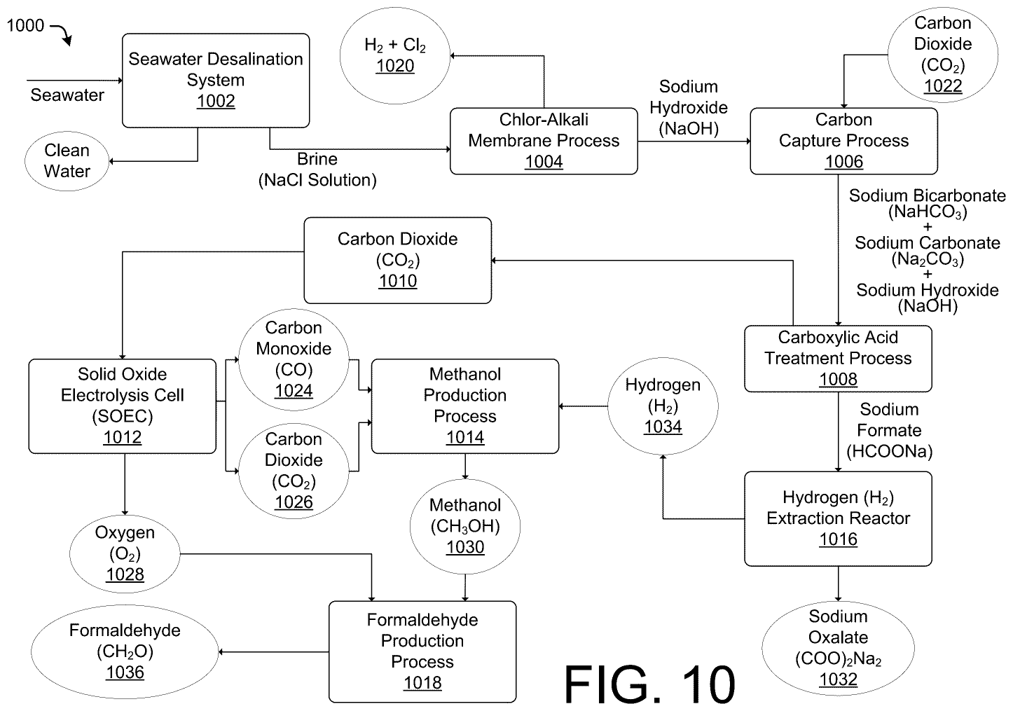

FIG. 10 illustrates a flow diagram of an example process 1000 associated with utilizing a Hydrogen (H₂) extraction reactor (e.g., the Hydrogen (H₂) extraction reactor 1016) and an electrochemical device (e.g., the SOEC 1012) for the simultaneous production of Sodium Oxalate ((COO)₂Na₂) and Formaldehyde (CH₂O). The example process 1000 may include the seawater desalination system 1002, the chlor-alkali membrane process 1004, the carbon capture process 1006, the carboxylic acid process 1008, the SOEC 1012, the Methanol production process 1014, the Hydrogen (H₂) extraction reactor 1016, and the Formaldehyde production process 1018. The process 1000 may include a closed-loop IES for the capturing and production of CO₂ and CO via an NaOH solution, reverse osmosis, and brine treatment for Methanol (CH₃OH) and subsequent Formaldehyde (CH₂O) productions.

In embodiments, the seawater desalination system 1002 may receive seawater and produce clean water and brine (e.g., a concentrated NaCl solution). The brine may be directed into the chlor-alkali membrane process 1004. The chlor-alkali membrane process 1004 may be configured to process the brine to produce Chlorine (Cl₂) and Hydrogen (H₂) 1020, and to regenerate clean water and produce Sodium Hydroxide (NaOH). The Sodium Hydroxide (NaOH) may be directed to the carbon capture process 1006 to be used for carbon capture. In embodiments the carbon capture process 1006 may receive Carbon Dioxide (CO₂) 1022 (e.g., atmospheric air that contains Carbon Dioxide (CO₂). The carbon capture process 1006 may utilize the Sodium Hydroxide (NaOH) produced by the chlor-alkali membrane process 1004 to capture Carbon Dioxide (CO₂) 1022 to produce Sodium Carbonate (Na₂CO₃) and Sodium Bicarbonate (NaHCO₃). In embodiments, the Sodium Carbonate (Na₂CO₃) and Sodium Bicarbonate (NaHCO₃) may be directed to the carboxylic acid treatment process 1008.

In embodiments, the carboxylic acid treatment process 1008 may include reacting the Sodium Carbonate (Na₂CO₃) and Sodium Bicarbonate (NaHCO₃) with a Carboxylic Acid (R—COOH) (e.g., Formic Acid (HCOOH), etc.) to produce Sodium Formate (HCOONa) and Carbon Dioxide (CO₂) 1010. The Carbon Dioxide (CO₂) 1010 may be directed to the SOEC 1012 to produce two gas streams. One gas stream (e.g., the first gas stream) may include a mixture of Carbon Monoxide (CO) 1024 and Carbon Dioxide (CO₂) 1026. The other gas stream (e.g., the second gas stream) may contain Oxygen (O₂) 1028. In embodiments, the Carbon Monoxide (CO) 1024 and the Carbon Dioxide (CO₂) 1026 may be used in the Methanol production process 1014 to produce Methanol (CH₃OH) 1030. The Hydrogen (H₂) extraction reactor 1016 may receive Sodium Formate (HCOONa) and produce Sodium Oxalate ((COO)₂Na₂) 1032 and Hydrogen (H₂) 1034. For example, the Sodium Formate (HCOONa) may be thermally decomposed to generate Sodium Oxalate ((COO)₂Na₂) 1032 and Hydrogen (H₂) 1034. In embodiments, the Hydrogen (H₂) 1034 may be directed to the Methanol production process 1014 to produce Methanol (CH₃OH) 1030. For example, the Hydrogen (H₂) may be used to react with the Carbon Monoxide (CO) to produce Methanol (CH₃OH) 1030.

In embodiments, the Methanol (CH₃OH) 1030 produced by the Methanol production process 1014 and the Oxygen (O₂) 1028 produced by the SOEC 1012 may be directed to the Formaldehyde production process 1018 to produce Formaldehyde (CH₂O) 1036. For example, Formaldehyde (CH₂O) 1036 may be produced by reacting the Methanol (CH₃OH) 1030 with the Oxygen (O₂) 1028 using moderate reaction temperatures.

For example, seawater undergoes the desalination process and is treated with brine (e.g., an NaCl solution) to produce Chlorine (Cl₂) and Hydrogen (H₂). In the process, clean water is extracted, and Sodium Hydroxide (NaOH) reacts with Carbon Dioxide (CO₂) (from direct air capture) to produce Sodium Carbonate (Na₂CO₃) and Sodium Bicarbonate (NaHCO₃). Carboxylic Acid (e.g., HCOOH) may react with the Sodium Carbonate (Na₂CO₃) and Sodium Bicarbonate (NaHCO₃) to produce Sodium Formate (HCOONa). Sodium Formate (HCOONa) may be heated to produce Sodium Oxalate ((COO)₂Na₂). The released Hydrogen (H₂) may react with Carbon Monoxide (CO) from an SOEC to produce Methanol (CH₃OH). Formaldehyde (CH₂O) may be produced by the of the reaction Methanol (CH₃OH) catalyzed by metals or metal oxides at moderate reaction temperatures.

In embodiments, Carboxylic Acid (HCOOH) reacts with the Sodium Carbonate (Na₂CO₃) and Sodium Bicarbonate (NaHCO₃) to regenerate Carbon Dioxide (CO₂). The Carbon Dioxide (CO₂) is fed into a SOEC to achieve the electrolysis of Carbon Dioxide (CO₂) by using a solid oxide, or ceramic, electrolyte to produce Carbon Monoxide (CO) and Oxygen (O₂).

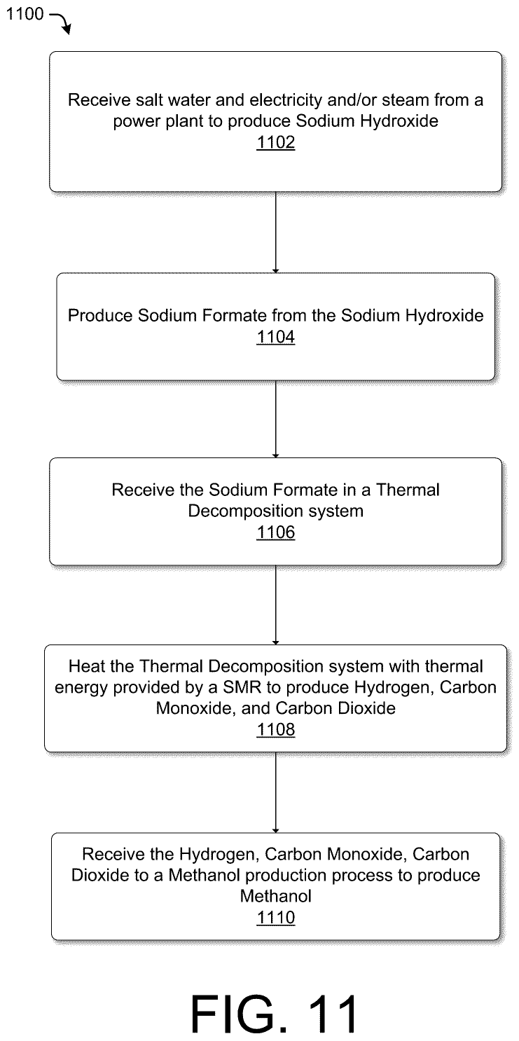

FIG. 11 illustrates a flowchart describing an example process 1100 for utilizing a power plant system to produce Methanol (CH₃OH). The order in which the operations or steps are described is not intended to be construed as a limitation, and any number of the described operations may be combined in any order and/or in parallel to implement the example process 1100.

At step 1102, the example process 1100 may include receiving salt water and electricity and/or steam from a power plant to produce Sodium Hydroxide (NaOH). In embodiments, the power plant includes at least one nuclear reactor and electrical power generation system, the at least one nuclear reactor being configured to generate the steam, and the electrical power generation system being configured to generate the electricity. In embodiments, the salt water is received to a water treatment plant, such as a Reverse Osmosis (RO) desalination process, configured to produce clean water and brine from the salt water, and wherein the Sodium Hydroxide (NaOH) is produced from the brine. In embodiments, the Sodium Hydroxide (NaOH) is produced from the salt water by an electrolysis process, such as a chlor-alkali process. In embodiments, the water treatment plant is configured to receive at least a portion of the steam and/or the electricity from the power plant. It is understood that the water treatment plant may be located in close relative proximity to the power plant (i.e., at the same site, etc.).

At step 1104, the example process 1100 may include producing Sodium Formate (HCOONa) from the Sodium Hydroxide (NaOH). In embodiments, the Sodium Hydroxide (NaOH) may be a received to a Sodium Formate (HCOONa) production process configured to produce Sodium Formate (HCOONa) from the Sodium Hydroxide (NaOH). In embodiments, the Sodium Formate (HCOONa) production process is configured to receive at least a portion of the steam and the electricity from the power plant. It is understood that the power plant may be at a location several miles from the Sodium Formate (HCOONa) production process. It is understood that the Sodium Formate (HCOONa) production process may be located in close relative proximity to the power plant (i.e., at the same site, etc.).

At step 1106, the example process 1100 may include receiving the Sodium Formate (HCOONa) in a Thermal Decomposition system. In embodiments, the Thermal Decomposition system may be configured to receive the Sodium Formate (HCOONa) and to produce Hydrogen (H₂), Carbon Dioxide (CO₂), and Carbon Monoxide (CO).

At step 1108, the example process 1100 may include heating the Thermal Decomposition system with thermal energy provided by a SMR to produce Hydrogen (H₂), Carbon Monoxide (CO), and Carbon Dioxide (CO₂). In embodiments, the Thermal Decomposition reactor is configured to receive at least a portion of the steam and/or the electricity from the power plant. In embodiments, the Thermal Decomposition reactor receives at least a portion of the steam as thermal energy to indirectly heat a Thermal Decomposition reactor. In embodiment, the Thermal Decomposition system receives the steam and/or electricity from the power plant to indirectly heat the Sodium Formate (HCOONa). In embodiments, the Thermal Decomposition reactor receives at least a portion of the steam to an interior of a Thermal Decomposition reactor to directly heat the Sodium Formate (HCOONa). In embodiments, the Thermal Decomposition system comprises a first chamber comprising an internal temperature between 300° C. and 450° C. and a second chamber comprising an internal temperature greater than 450° C.

In embodiments, the Thermal Decomposition system includes a first chamber configured to produce the Hydrogen (H₂), the Carbon Monoxide (CO), and Sodium Carbonate (Na₂CO₃), including a first rotating spiral configured to direct the Sodium Formate (HCOONa) into an upper portion of the first chamber, and a second rotating spiral configured to direct the Sodium Carbonate (Na₂CO₃) out of a lower portion of the first chamber. In embodiments, the Thermal Decomposition System includes a second chamber configured to receive the Sodium Carbonate (Na₂CO₃) from the second rotating spiral and produce the Carbon Dioxide (CO₂) and Sodium Oxide (Na₂O) including a third rotating spiral configured to direct the Sodium Carbonate (Na₂CO₃) into an upper portion of the second chamber, and a fourth rotating spiral configured to direct the Sodium Oxide (Na₂O) out of a lower portion of the second chamber. It is understood that the power plant may be at a location several miles from the Thermal Decomposition system. It is understood that the Thermal Decomposition system may be located in close relative proximity to the power plant (i.e., at the same site, etc.).

At step 1110, the example process 1100 may include receiving the Hydrogen (H₂), Carbon Dioxide (CO₂), and Carbon Monoxide (CO) to a Methanol production process to produce Methanol (CH₃OH). In embodiments, the Methanol (CH₃OH) production process is configured to receive at least a portion of the steam and/or the electricity from the power plant. It is understood that the power plant may be at a location several miles from the Methanol (CH₃OH) production process. It is understood that the Methanol (CH₃OH) production process may be located in close relative proximity to the power plant (i.e., at the same site, etc.).

FIGS. 12 and 13 illustrate representative nuclear reactors that may be included in embodiments of the present technology. FIG. 12 is a partially schematic, partially cross-sectional view of a nuclear reactor system 1200 configured in accordance with embodiments of the present technology. The system 1200 can include a power module 1202 having a reactor core 1204 in which a controlled nuclear reaction takes place. Accordingly, the reactor core 1204 can include one or more fuel assemblies 1201. The one or more fuel assemblies 1201 can include fissile and/or other suitable materials. Heat from the reaction generates steam at a steam generator 1230, which directs the steam to a power conversion system 1240. The power conversion system 1240 generates electrical power, and/or provides other useful outputs, such as super-heated steam. A sensor system 1250 is used to monitor the operation of the power module 1202 and/or other system components. The data obtained from the sensor system 1250 can be used in real time to control the power module 1202, and/or can be used to update the design of the power module 1202 and/or other system components.

The power module 1202 includes a containment vessel 1210 (e.g., a radiation shield vessel, or a radiation shield container) that houses/encloses a reactor vessel 1220 (e.g., a reactor pressure vessel, or a reactor pressure container), which in turn houses the reactor core 1204. The containment vessel 1210 can be housed in a power module bay 1256. The power module bay 1256 can contain a cooling pool 1203 filled with water and/or another suitable cooling liquid. The bulk of the power module 1202 can be positioned below a surface 1205 of the cooling pool 1203. Accordingly, the cooling pool 1203 can operate as a thermal sink, for example, in the event of a system malfunction.

A volume between the reactor vessel 1220 and the containment vessel 1210 can be partially or completely evacuated to reduce heat transfer from the reactor vessel 1220 to the surrounding environment (e.g., to the cooling pool 1203). However, in other embodiments the volume between the reactor vessel 1220 and the containment vessel 1210 can be at least partially filled with a gas and/or a liquid that increases heat transfer between the reactor vessel 1220 and the containment vessel 1210. For example, the volume between the reactor vessel 1220 and the containment vessel 1210 can be at least partially filled (e.g., flooded with the primary coolant 1207) during an emergency operation.

Within the reactor vessel 1220, a primary coolant 1207 conveys heat from the reactor core 1204 to the steam generator 1230. For example, as illustrated by arrows located within the reactor vessel 1220, the primary coolant 1207 is heated at the reactor core 1204 toward the bottom of the reactor vessel 1220. The heated primary coolant 1207 (e.g., water with or without additives) rises from the reactor core 1204 through a core shroud 1206 and to a riser tube 1208. The hot, buoyant primary coolant 1207 continues to rise through the riser tube 1208, then exits the riser tube 1208 and passes downwardly through the steam generator 1230. The steam generator 1230 includes a multitude of conduits 1232 that are arranged circumferentially around the riser tube 1208, for example, in a helical pattern, as is shown schematically in FIG. 12. The descending primary coolant 1207 transfers heat to a secondary coolant (e.g., water) within the conduits 1232, and descends to the bottom of the reactor vessel 1220 where the cycle begins again. The cycle can be driven by the changes in the buoyancy of the primary coolant 1207, thus reducing or eliminating the need for pumps to move the primary coolant 1207.

The steam generator 1230 can include a feedwater header 1231 at which the incoming secondary coolant enters the steam generator conduits 1232. The secondary coolant rises through the conduits 1232, converts to vapor (e.g., steam), and is collected at a steam header 1233. The steam exits the steam header 1233 and is directed to the power conversion system 1240.

The power conversion system 1240 can include one or more steam valves 1242 that regulate the passage of high pressure, high temperature steam from the steam generator 1230 to a steam turbine 1243. The steam turbine 1243 converts the thermal energy of the steam to electricity via a generator 1244. The low-pressure steam exiting the turbine 1243 is condensed at a condenser 1245, and then directed (e.g., via a pump 1246) to one or more feedwater valves 1241. The feedwater valves 1241 control the rate at which the feedwater re-enters the steam generator 1230 via the feedwater header 1231. In other embodiments, the steam from the steam generator 1230 can be routed for direct use in an industrial process, such as a Hydrogen (H₂) and Oxygen (O₂) production plant, a chemical production plant, and/or the like, as described in detail below. Accordingly, steam exiting the steam generator 1230 can bypass the power conversion system 1240.

The power module 1202 includes multiple control systems and associated sensors. For example, the power module 1202 can include a hollow cylindrical reflector 1209 that directs neutrons back into the reactor core 1204 to further the nuclear reaction taking place therein. Control rods 1213 are used to modulate the nuclear reaction and are driven via fuel rod drivers 1215. The pressure within the reactor vessel 1220 can be controlled via a pressurizer plate 1217 (which can also serve to direct the primary coolant 1207 downwardly through the steam generator 1230) by controlling the pressure in a pressurizing volume 1219 positioned above the pressurizer plate 1217.

The sensor system 1250 can include one or more sensors 1251 positioned at a variety of locations within the power module 1202 and/or elsewhere, for example, to identify operating parameter values and/or changes in parameter values. The data collected by the sensor system 1250 can then be used to control the operation of the system 1200, and/or to generate design changes for the system 1200. For sensors positioned within the containment vessel 1210, a sensor link 1252 directs data from the sensors to a flange 1253 (at which the sensor link 1252 exits the containment vessel 1210) and directs data to a sensor junction box 1254. From there, the sensor data can be routed to one or more controllers and/or other data systems via a data bus 1255.

FIG. 13 is a partially schematic, partially cross-sectional view of a nuclear reactor system 1300 configured in accordance with additional embodiments of the present technology. In some embodiments, the nuclear reactor system 1300 (“system 1300”) can include some features that are at least generally similar in structure and function, or identical in structure and function, to the corresponding features of the system 1300 described in detail above with reference to FIG. 13 and can operate in a generally similar or identical manner to the system 1300.

In the illustrated embodiment, the system 1300 includes a reactor vessel 1320 and a containment vessel 1310 surrounding/enclosing the reactor vessel 1320. In some embodiments, the reactor vessel 1320 and the containment vessel 1310 can be roughly cylinder-shaped or capsule-shaped. The system 1300 further includes a plurality of heat pipe layers 1311 within the reactor vessel 1320. In the illustrated embodiment, the heat pipe layers 1311 are spaced apart from and stacked over one another. In some embodiments, the heat pipe layers 1311 can be mounted/secured to a common frame 1312, a portion of the reactor vessel 1320 (e.g., a wall thereof), and/or other suitable structures within the reactor vessel 1320. In other embodiments, the heat pipe layers 1311 can be directly stacked on top of one another such that each of the heat pipe layers 1311 supports and/or is supported by one or more of the other ones of the heat pipe layers 1311.

In the illustrated embodiment, the system 1300 further includes a shield or reflector region 1314 at least partially surrounding a core region 1316. The heat pipe layers 1311 can be circular, rectilinear, polygonal, and/or can have other shapes, such that the core region 1316 has a corresponding three-dimensional shape (e.g., cylindrical, spherical). In some embodiments, the core region 1316 is separated from the reflector region 1314 by a core barrier 1315, such as a metal wall. The core region 1316 can include one or more fuel sources, such as fissile material, for heating the heat pipe layers 1311. The reflector region 1314 can include one or more materials configured to contain/reflect products generated by burning the fuel in the core region 1316 during operation of the system 1300. For example, the reflector region 1314 can include a liquid or solid material configured to reflect neutrons and/or other fission products radially inward toward the core region 1316. In some embodiments, the reflector region 1314 can entirely surround the core region 1316. In other embodiments, the reflector region 1314 may partially surround the core region 1316. In some embodiments, the core region 1316 can include a control material 1317, such as a moderator and/or coolant. The control material 1317 can at least partially surround the heat pipe layers 1311 in the core region 1316 and can transfer heat therebetween.

In the illustrated embodiment, the system 1300 further includes at least one heat exchanger 1330 (e.g., a steam generator) positioned around the heat pipe layers 1311. The heat pipe layers 1311 can extend from the core region 1316 and at least partially into the reflector region 1314 and are thermally coupled to the heat exchanger 1330. In some embodiments, the heat exchanger 1330 can be positioned outside of or partially within the reflector region 1314. The heat pipe layers 1311 provide a heat transfer path from the core region 1316 to the heat exchanger 1330. For example, the heat pipe layers 1311 can each include an array of heat pipes that provide a heat transfer path from the core region 1316 to the heat exchanger 1330. When the system 1300 operates, the fuel in the core region 1316 can heat and vaporize a fluid within the heat pipes in the heat pipe layers 1311, and the fluid can carry the heat to the heat exchanger 1330. The heat pipes in the heat pipe layers 1311 can then return the fluid toward the core region 1316 via wicking, gravity, and/or other means to be heated and vaporized once again.

In some embodiments, the heat exchanger 1330 can be similar to the steam generator 1230 of FIG. 12 and, for example, can include one or more helically-coiled tubes that wrap around the heat pipe layers 1311. The tubes of the heat exchanger 1330 can include or carry a working fluid (e.g., a coolant such as water or another fluid) that carries the heat from the heat pipe layers 1311 out of the reactor vessel 1320 and the containment vessel 1310 for use in generating electricity, steam, and/or the like. For example, in the illustrated embodiment the heat exchanger 1330 is operably coupled to a turbine 1343, a generator 1344, a condenser 1345, and a pump 1346. As the working fluid within the heat exchanger 1330 increases in temperature, the working fluid may begin to boil and vaporize. The vaporized working fluid (e.g., steam) may be used to drive the turbine 1343 to convert the thermal potential energy of the working fluid into electrical energy via the generator 1344. The condenser 1345 can condense the working fluid after it passes through the turbine 1343, and the pump 1346 can direct the working fluid back to the heat exchanger 1330 where it can begin another thermal cycle. In other embodiments, steam from the heat exchanger 1330 can be routed for direct use in an industrial process, such as an enhanced oil recovery operation described in detail below. Accordingly, steam exiting the heat exchanger 1330 can bypass the turbine 1343, the generator 1344, the condenser 1345, the pump 1346, etc.

FIG. 14 is a schematic view of a nuclear power plant system 1450 including multiple nuclear reactors 1400 in accordance with embodiments of the present technology. Each of the nuclear reactors 1400 (individually identified as first through twelfth nuclear reactors 1400a-l, respectively) can be similar to or identical to the nuclear reactors 1400 and/or the nuclear reactors 1400 described in detail above with reference to FIGS. 12 and 13. The power plant system 1450 (“power plant system 1450”) can be “modular” in that each of the nuclear reactors 1400 can be operated separately to provide an output, such as electricity or steam. The power plant system 1450 can include fewer than twelve of the nuclear reactors 1400 (e.g., two, three, four, five, six, seven, eight, nine, ten, or eleven of the nuclear reactors 1400), or more than twelve of the nuclear reactors 1400. The power plant system 1450 can be a permanent installation or can be mobile (e.g., mounted on a truck, tractor, mobile platform, and/or the like). In the illustrated embodiment, each of the nuclear reactors 1400 can be positioned within a common housing 1451, such as a reactor plant building, and controlled and/or monitored via a control room 1452.

Each of the nuclear reactors 1400 can be coupled to a corresponding electrical power conversion system 1440 (individually identified as first through twelfth electrical power conversion systems 1440a-l, respectively). The electrical power conversion systems 1440 can include one or more devices that generate electrical power or some other form of usable power from steam generated by the nuclear reactors 1400. In some embodiments, multiple ones of the nuclear reactors 1400 can be coupled to the same one of the electrical power conversion systems 1440 and/or one or more of the nuclear reactors 1400 can be coupled to multiple ones of the electrical power conversion systems 1440 such that there is not a one-to-one correspondence between the nuclear reactors 1400 and the electrical power conversion systems 1440.

The electrical power conversion systems 1440 can be further coupled to an electrical power transmission system 1454 via, for example, an electrical power bus 1453. The electrical power transmission system 1454 and/or the electrical power bus 1453 can include one or more transmission lines, transformers, and/or the like for regulating the current, voltage, and/or other characteristic(s) of the electricity generated by the electrical power conversion systems 1440. The electrical power transmission system 454 can route electricity via a plurality of electrical output paths 1455 (individually identified as electrical output paths 1455a-n) to one or more end users and/or end uses, such as different electrical loads of an integrated energy system.

Each of the nuclear reactors 1400 can further be coupled to a steam transmission system 1456 via, for example, a steam bus 1457. The steam bus 1457 can route steam generated from the nuclear reactors 1400 to the steam transmission system 1456 which in turn can route the steam via a plurality of steam output paths 1458 (individually identified as steam output paths 1458a-n) to one or more end users and/or end uses, such as different steam inputs of an integrated energy system.

In some embodiments, the nuclear reactors 1400 can be individually controlled (e.g., via the control room 1452) to provide steam to the steam transmission system 1456 and/or steam to the corresponding one of the electrical power conversion systems 1440 to provide electricity to the electrical power transmission system 1454. In some embodiments, the nuclear reactors 1400 are configured to provide steam either to the steam bus 1457 or to the corresponding one of the electrical power conversion systems 1440 and can be rapidly and efficiently switched between providing steam to either. Accordingly, in some aspects of the present technology the nuclear reactors 1400 can be modularly and flexibly controlled such that the power plant system 1450 can provide differing levels/amounts of electricity via the electrical power transmission system 1454 and/or steam via the steam transmission system 1456. For example, where the power plant system 1450 is used to provide electricity and steam to one or more industrial process-such as various components of the integrated energy systems, the nuclear reactors 1400 can be controlled to meet the differing electricity and steam requirements of the industrial processes.

As one example, during a first operational state of an integrated energy system employing the power plant system 1450, a first subset of the nuclear reactors 1400 (e.g., the first through sixth nuclear reactors 1400a-f) can be configured to provide steam to the steam transmission system 1456 for use in the first operational state of the integrated energy system, while a second subset of the nuclear reactors 1400 (e.g., the seventh through twelfth nuclear reactors 1400g-l) can be configured to provide steam to the corresponding ones of the electrical power conversion systems 1440 (e.g., the seventh through twelfth electrical power conversion systems 1440g-l) to generate electricity for the first operational state of the integrated energy system. Then, during a second operational state of the integrated energy system when a different (e.g., greater or lesser) amount of steam and/or electricity is required, some or all the first subset of the nuclear reactors 1400 can be switched to provide steam to the corresponding ones of the electrical power conversion systems 1440 (e.g., the seventh through twelfth electrical power conversion systems 1440g-l) and/or some or all of the second subset of the nuclear reactors 1400 can be switched to provide steam to the steam transmission system 1456 to vary the amount of steam and electricity produced to match the requirements/demands of the second operational state. Other variations of steam and electricity generation are possible based on the needs of the integrated energy system. That is, the nuclear reactors 1400 can be dynamically/flexibly controlled during other operational states of an integrated energy system to meet the steam and electricity requirements of the operational state.

In contrast, some conventional nuclear power plant systems can typically generate either steam or electricity for output and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 1400 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured, or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

### CONCLUSION

During each of the representative production processes described above, Carbon Dioxide (CO₂) generation (e.g., carbon footprints) are either drastically reduce or eliminated. The end results may produce a greener world for our children and grandchildren for many generations to follow.

Although several embodiments have been described in language specific to structural features and/or methodological acts, it is to be understood that the claims are not necessarily limited to the specific features or acts described. Rather, the specific features and acts are disclosed as illustrative forms of implementing the claimed subject matter.

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments. Embodiments of the technology disclosed herein may be applied to systems other than those expressly described herein. For example, the technology can be applied to other steam generators with boiling on the inside of tubes, heat exchangers or processing equipment with flow into multiple pipes, and/or similar fluid handling devices that may exhibit density-wave oscillations with potential to cause excessive thermal cycling stresses at the tube or pipe inlets.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. An Integrated Energy System (IES) comprising:
a power plant configured to generate steam and electricity;
a water treatment plant to produce Sodium Hydroxide (NaOH) from salt water;
a Sodium Formate (HCOONa) production plant configured to receive the Sodium Hydroxide (NaOH) to produce Sodium Formate (HCOONa);
a Thermal Decomposition reactor configured to receive the Sodium Formate (HCOONa) and produce Hydrogen (H₂), Carbon Dioxide (CO₂), and Carbon Monoxide (CO); and
a Methanol (CH₃OH) reaction chamber configured to receive the Hydrogen (H₂), the Carbon Dioxide (CO₂), and the Carbon Monoxide (CO) to produce Methanol (CH₃OH).

2. The IES of claim 1, wherein the power plant comprises at least one nuclear reactor and electrical power generation system, the at least one nuclear reactor being configured to generate the steam, and the electrical power generation system being configured to generate the electricity.

3. The IES of claim 1, wherein the water treatment plant is configured to receive at least a first portion of the steam or at least a second portion of the electricity from the power plant.

4. The IES of claim 1, wherein the water treatment plant is a desalination process configured to produce clean water and brine from the salt water, and wherein the Sodium Hydroxide (NaOH) is produced from the brine.

5. The IES of claim 4, wherein the desalination process comprises reverse osmosis.

6. The IES of claim 1, wherein the Sodium Hydroxide (NaOH) is produced from the salt water by an electrolysis process.

7. The IES of claim 1, wherein the Sodium Formate (HCOONa) production plant is configured to receive at least a first portion of the steam or at least a second portion of the electricity from the power plant.

8. The IES of claim 1, wherein the Thermal Decomposition reactor is configured to receive at least a first portion of the steam or at least a second portion of the electricity from the power plant.

9. The IES of claim 8, wherein the Thermal Decomposition reactor receives at least a portion of the steam as thermal energy to indirectly heat the Thermal Decomposition reactor.

10. The IES of claim 1, wherein the Methanol (CH₃OH) reaction chamber is configured to receive at least a first portion of the steam or at least a second portion of the electricity from the power plant.

11. An Integrated Energy System (IES) comprising:
a power plant configured to generate steam and electricity;
a Thermal Decomposition reactor configured to receive at least a first portion of the steam or at least a second portion of the electricity from the power plant to indirectly heat the Thermal Decomposition reactor to produce Hydrogen (H₂), Carbon Dioxide (CO₂), and Carbon Monoxide (CO) from Sodium Formate (HCOONa); and
a Methanol (CH₃OH) reaction chamber configured to receive the Hydrogen (H₂), the Carbon Dioxide (CO₂), and the Carbon Monoxide (CO) to produce Methanol (CH₃OH).

12. The IES of claim 11, wherein the power plant comprises at least one nuclear reactor and electrical power generation system, the at least one nuclear reactor being configured to generate the steam, and the electrical power generation system being configured to generate the electricity.

13. The IES of claim 11, wherein the Thermal Decomposition reactor comprises:
a first chamber configured to produce the Hydrogen (H₂), the Carbon Monoxide (CO), and Sodium Carbonate (Na₂CO₃), comprising:
a first rotating spiral configured to direct the Sodium Formate (HCOONa) into an upper portion of the first chamber, and
a second rotating spiral configured to direct the Sodium Carbonate (Na₂CO₃) out of a lower portion of the first chamber; and
a second chamber configured to receive the Sodium Carbonate (Na₂CO₃) from the second rotating spiral and produce the Carbon Dioxide (CO₂) and Sodium Oxide (Na₂O) comprising:
a third rotating spiral configured to direct the Sodium Carbonate (Na₂CO₃) into an upper portion of the second chamber, and
a fourth rotating spiral configured to direct the Sodium Oxide (Na₂O) out of a lower portion of the second chamber.

14. The IES of claim 11, wherein the Methanol (CH₃OH) reaction chamber is configured to receive at least a first portion of the steam or at least a second portion of the electricity from the power plant.

15. A method comprising:
receiving salt water and electricity from a power plant to a water treatment process to produce Sodium Hydroxide (NaOH);
receiving the Sodium Hydroxide (NaOH) into a Sodium Formate (HCOONa) production plant to produce Sodium Formate (HCOONa);
receiving the Sodium Formate (HCOONa) into a Thermal Decomposition reactor to produce Hydrogen (H₂), Carbon Dioxide (CO₂), and Carbon Monoxide (CO) from the Sodium Formate (HCOONa); and
combining the Hydrogen (H₂), the Carbon Dioxide (CO₂), and the Carbon Monoxide (CO) in a Methanol (CH₃OH) production plant to produce Methanol (CH₃OH).

16. The method of claim 15, wherein the power plant comprises at least one nuclear reactor and electrical power generation system, the at least one nuclear reactor being configured to generate steam, and the electrical power generation system being configured to generate the electricity.

17. The method of claim 15, further comprising receiving at least a first portion of the steam or at least a second portion of the electricity from the power plant to the Thermal Decomposition reactor to indirectly heat the Sodium Formate (HCOONa).

18. The method of claim 15, further comprising receiving steam from the power plant to an interior of the Thermal Decomposition reactor to directly heat the Sodium Formate (HCOONa).

19. The method of claim 15, wherein the Thermal Decomposition reactor comprises a first chamber comprising an internal temperature between 300° C. and 450° C. and a second chamber comprising an internal temperature greater than 450° C.

20. The method of claim 19, wherein the first chamber receives the Sodium Formate (HCOONa) and produces the Hydrogen (H₂), the Carbon Monoxide (CO), and Sodium Carbonate (Na₂CO₃) from thermal decomposition of the Sodium Formate (HCOONa), and the second chamber receives the Sodium Carbonate (Na₂CO₃) from the first chamber and produces the Carbon Dioxide (CO₂) and Sodium Oxide (Na₂O) from thermal decomposition of the Sodium Carbonate (Na₂CO₃).
